(19) United States
(12) Patent Application Publication
(10) Pub. No.: US 2011/0081698 A1
(43) Pub. Date: Apr. 7, 2011

(54) METHOD AND INTRODUCTION OF GENE INTO YEAST CELL, AND VECTOR FOR THE METHOD

(75) Inventors: Hideo Noda, Hyogo (JP); Shohci Kaneko, Hyogo (JP); Akihiko Kondo, Hyogo (JP)

(73) Assignees: BIO-ENERGAY CORPORATION, HYOGO (JP); KANSAI CHEMICAL ENGINEERING CO., LTD., HYOGO (JP)

(21) Appl. No.: 12/992,118

(22) PCT Filed: May 11, 2009
(86) PCT No.: PCT/JP2009/058765

§ 371 Date: Nov. 11, 2010

(30) Foreign Application Priority Data
May 14, 2008 (JP) 2008-126720

(51) Int. Cl. C12P 7/10; C12N 15/81; C12N 1/19

(52) U.S. Cl. 435/165; 435/471; 435/320.1; 435/254.2

(57) ABSTRACT

An object of the present invention is a method for introducing a foreign gene into a yeast cell that does not have an auxotrophic marker. The present invention provides a method for providing a target auxotrophy to a yeast cell and introducing a gene to be expressed into the yeast cell. The method includes the step of transforming a yeast cell with a fragment containing an expression cassette for the gene to be expressed, a cassette for a yeast selectable marker, and two homologous recombination fragments each homologous to a region on either side of a target auxotrophy controlling gene. According to the method, a target auxotrophy controlling gene is deleted from a yeast cell and a gene to be expressed is introduced into the yeast cell, and further the yeast selectable marker is eliminated from the transformed yeast cell.

### TECHNICAL FIELD

The present invention relates to a method for introducing a gene into a yeast cell.

### BACKGROUND ART

The 21st century has been facing increasingly serious resource and environmental problems. Arising from the issues of the depletion of fossil resources such as petroleum, generous use of biomass has been major challenges toward implementation of renewable resources, energy saving, and reduction in greenhouse gases.

In addition, it is necessary to stop the one-way system of producing large amounts of goods from limited resources and consuming and disposing of large amounts of those goods, and convert the industrial structure into a form that enables sustainable development.

Effective use of biomass resources that are not yet in use has been attracting attention to address such issues. One example is the development of a process for conversion into bioethanol. Bioethanol is ethanol obtained from biomass. In this regard, wood-based biomass resources, which have been used as energy by direct combustion since recorded history, are liquefied into the form of ethanol by microorganisms such as yeasts, and then used as energy. It is thus thought that the use of wood-based biomass as an energy source will be broadened such as enhancement of energy conversion efficiency and transportability.

Yeast Saccharomyces cerevisiae has been used in the production of various chemical substances and the production of ethanol for use as fuel. Ethanol is now mostly made from starch (amylose). Yeasts do not have any enzymes capable of degrading amylose and thus cannot degrade starch. Therefore, at the time, starch is acted with amylase while heating to degrade it to glucose, and the resulting glucose is fermented by a yeast to produce ethanol. Also, cellulose, which is a principal component of agricultural wastes and waste paper, is acted with cellulose to generate glucose as a degradation product. Yeasts do not have any enzymes capable of degrading amylose or cellulose. If yeasts acquire such an enzyme, the two-stage process of saccharification and fermentation can be performed in one step.

A yeast that allows for ethanol fermentation directly from carbohydrates has been created through the preparation of yeast Saccharomyces cerevisiae that can express to display on the cell surface or secrete, an enzyme such as an amylose-degrading enzymes or an cellulose-degrading enzyme. Accordingly, a yeast that enables ethanol fermentation from amylose or cellulose has been successfully created (Patent Documents 1 to 3). The yeast Saccharomyces cerevisiae cannot metabolize xylose. The yeast Saccharomyces cerevisiae that displays on the cell surface xylan-degrading enzymes xylanase 2 (XYNII) derived from Trichoderma reesei and β-xylosidase (XylA) derived from Aspergillus oryzae and that expresses a xylose reductase (XR) gene and a xylitol dehydrogenase (XDH) gene (both derived from Pichia stipitis) and a xylulokinase (XK) gene (derived from Saccharomyces cerevisiae) has been produced, and attempts have been made to produce ethanol from xylan in birches using this yeast (Non-Patent Document 1).

Patent Documents 1 to 3 and Non-Patent Document 1 disclose the effects of recombinant yeasts from a Saccharomyces cerevisiae MT8-1 strain as a host. Since the MT8-1 strain has five different auxotrophies and is easy to deal with for various gene manipulations, it has been used for the creation of yeasts having various functions. However, the MT8-1 strain is poor in alcohol fermentation ability, alcohol resistance, acid resistance, heat resistance, and the like, and thus is not preferable for industrial use.

Industrial yeasts are superior in alcohol fermentation ability, alcohol resistance, acid resistance, and heat resistance, and thus are desirable in alcohol production. However, industrial yeasts do not have an auxotrophic marker that is necessary in performing gene manipulation, making it difficult to perform gene manipulation.

For these industrial yeasts, dominant drug-resistant markers are useful because such markers do not require any mutations in the host. However, these drug-resistant markers have several disadvantages. Transformation efficiencies of the drug-resistant markers are usually lower than those of auxotrophic markers. Transformant colonies selected by drug resistance are usually contaminated with nontransformed colonies that are a result of spontaneous drug resistance mutation or cells that are more resistant than the majority of the populations. It is also often used that the method for deleting a bacterial hisG or loxP sequence put on a counter selectable marker gene flanked by a specific recombinase is also often used. However, reduction in transformation efficiencies is caused by using this method many times because this method leaves one copy of the hisG or loxP sequence on chromosome (Non-Patent Document 2).

A marker recycling method that uses PCR-mediated seamless gene deletion was developed to avoid this problem (Non-Patent Document 3). Non-Patent Document 3 discloses that, using the BY4740 (MATa) and BY4743 (MATα) strains of Saccharomyces cerevisiae, crossover between His3 on a yeast chromosome and a fragment containing URA3 and a sequence homologous to the sequence in vicinity to His3 on the yeast chromosome is made to delete His3 from the chromosome, and then, the homologous recombination is caused by use of repeat sequences in the yeast chromosome to recover URA3 without left on the yeast chromosome. This marker recycling method can delete a gene without leaving any extra gene sequence or recycling marker in host genome. In the molecular breeding of an industrial yeast, this technique is useful but is required vast time in sequential gene disruption, and also limits to acquire any auxotrophic markers.

### Prior Art Document

### PATENT DOCUMENT

[Patent Document 1] WO 02/42483
[Patent Document 2] WO 03/016525
[Patent Document 3] WO 01/79483

### Non-Patent Document

[Non-Patent Document 1] S. Katahira et al., Applied and Environmental Microbiology, 2004, vol. 70, pp. 5407-5414
[Non-Patent Document 2] Davidson and Schiestl, Curr. Genet., 2000, vol. 38, pp. 188-190
[Non-Patent Document 3] R. Akada et al., Yeast, 2006, vol. 23, pp. 399-405
[Non-Patent Document 4] Appl. Microbiol. Biotech., 2002, vol. 60, pp. 469-474
[Non-Patent Document 5] Applied and Environmental Microbiology 2002, vol. 68, pp. 4517-4522
[Non-Patent Document 6] Tajima et al., Yeast, 1985, vol. 1, pp. 67-77
[Non-Patent Document 7] Rose et al., Methods in Yeast Genetics, A Laboratory Course Manual, Cold Spring Harbor Laboratory Press: Cold Spring Harbor, N.Y., 1990
[Non-Patent Document 8] Akada et al., Biotechniques, 2000, vol. 28, pp. 668-70, 672, 674
[Non-Patent Document 9] P. N. Lipke et al., Mol. Cell. Biol., August 1989, 9(8), pp. 3155-65
[Non-Patent Document 10] Y. Fujita et al., Applied and Environmental Microbiology, 2002, vol. 68, pp. 5136-41
[Non-Patent Document 11] Fujita et al., Appl. Environ. Microbiol, 2004, vol. 20, pp. 1207-1212
[Non-Patent Document 12] Takahashi et al., Appl. Microbiol. Biotechnol., 2001, vol. 55, pp. 454-462

### SUMMARY OF THE INVENTION

### Problems to be solved by the Invention

An object of the present invention is to provide a method for introducing a foreign gene into a yeast cell that does not have an auxotrophic marker.

### Means for Solving the Problems

The present invention provides a method for providing a target auxotrophy to and introducing a gene to be expressed into a yeast cell, the method comprising:

(a) transforming, with a fragment comprising an expression cassette for the gene to be expressed, a cassette for a yeast selectable marker that controls an auxotrophy different from the target auxotrophy and that allows counter selection, and two regions for homologous recombination, a yeast cell to which a marker of the different auxotrophy is provided,

wherein, in the fragment,

an upstream-side one of the homologous recombination regions is homologous to a region upstream (on the side of 5′ end) of a gene controlling the target auxotrophy in the yeast cell and is arranged upstream (on the side of 5′ end) of the expression cassette, and a downstream-side one of the homologous recombination regions is homologous to a region downstream (on the side of 3′ end) of the gene controlling the target auxotrophy and is arranged downstream (on the side of 3′ end) of the expression cassette,

the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the gene controlling the target auxotrophy, and

the yeast selectable marker cassette is arranged between the expression cassette and the downstream-side one of the homologous recombination region,

thereby causing a first homologous recombination;

(b) selecting for a transformed yeast not having the different auxotrophy, the transformed yeast from which the gene controlling the target auxotrophy is deleted and into which the gene to be expressed and the yeast selectable marker cassette is introduced;

(c) causing a second homologous recombination, in the transformed yeast selected in step (b), between the repeat region and the region further downstream (on the side of 3′ end) of the region downstream (on the side of 3′ end) of the gene controlling the target auxotrophy; and

(d) selecting for a transformed yeast that has acquired the different auxotrophy, to obtain a transformed yeast from which the gene controlling the target auxotrophy and the gene controlling the different auxotrophy are deleted, and which has an auxotrophy therefor, and into which the gene to be expressed is introduced.

In one embodiment, the method further comprises providing the different auxotrophy to the yeast cell.

The present invention further provides a method for repetitively introducing a gene into a yeast cell, the method comprising:

further transforming a transformed yeast cell produced according to the method for providing a target auxotrophy to and introducing a gene to be expressed into a yeast cell as mentioned above, from which an auxotrophy-controlling gene is deleted and into which a gene to be expressed is introduced, with a vector comprising the auxotrophy-controlling gene deleted from the transformed yeast cell and an additional gene to be expressed.

The present invention also provides a vector for providing a target auxotrophy to and introducing a gene to be expressed into a yeast cell, the vector comprising:

a fragment comprising an expression cassette for the gene, a cassette for a yeast selectable marker that is a gene controlling an auxotrophy different from the target auxotrophy, and two regions for homologous recombination,

wherein, in the fragment,

an upstream-side one of the homologous recombination regions being homologous to a region upstream (on the side of 5′ end) of a gene controlling the target auxotrophy in the yeast cell and is arranged upstream (on the side of 5′ end) of the expression cassette, and a downstream-side one of the homologous recombination regions is homologous to a region downstream (on the side of 3′ end) of the gene controlling the target auxotrophy and is arranged downstream (on the side of 3′ end) of the expression cassette,

the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the gene controlling the target auxotrophy, and

the yeast selectable marker cassette is arranged between the expression cassette and the downstream-side one of the homologous recombination regions.

In one embodiment of the method and vector, the different auxotrophy gene marker is uracil auxotrophy.

Further, the present invention provides a method for introducing a gene to be expressed into a yeast cell, the method comprising:

(i) transforming the yeast cell with a fragment comprising an expression cassette for the gene to be expressed, a cassette for a yeast selectable marker, and two regions for homologous recombination,

wherein, in the fragment,

an upstream-side one of the homologous recombination regions is homologous to a region upstream (on the side of 5′ end) of a target locus in the yeast cell and is arranged upstream (on the side of 5′ end) of the expression cassette, and a downstream-side one of the homologous recombination regions is homologous to a region downstream (on the side of 3′ end) of the target locus and is arranged downstream (on the side of 3′ end) of the expression cassette,

the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the target locus, and

the yeast selectable marker cassette is arranged between the expression cassette and the downstream-side one of the homologous recombination regions,

thereby causing a first homologous recombination, and wherein while the target locus is deleted, the gene to be expressed and the yeast selectable marker cassette is introduced into the yeast cell; and

(ii) causing a second homologous recombination between the region further downstream (on the side of 3′ end) of the region downstream (on the side of 3′ end) of the target locus and the repeat region to delete the yeast selectable marker from the transformed yeast cell.

The present invention provides a vector for introducing a gene to be expressed into a yeast cell, the vector comprising:

a fragment comprising an expression cassette for the gene to be expressed, a cassette for a yeast selectable marker, and two regions for homologous recombination,

wherein in the fragment,

an upstream-side one of the homologous recombination regions is homologous to a region upstream (on the side of 5′ end) of a target locus in the yeast cell and is arranged upstream (on the side of 5′ end) of the expression cassette, and a downstream-side one of the homologous recombination regions is homologous to a region downstream (on the side of 3′ end) of the target locus and is arranged downstream (on the side of 3′ end) of the expression cassette,

the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the target locus, and

the yeast selectable marker cassette is arranged between the expression cassette and the downstream-side one of the homologous recombination regions.

The present invention further provides a cellulolytic industrial yeast recombined to express an enzyme that can cleave a β-1,4-glycosidic bond.

In one embodiment of the cellulolytic industrial yeast, the enzyme that can cleave a β-1,4-glycosidic bond is a combination of β-glucosidase, endoglucanase, and cellobiohydrolase.

The present invention also provides a method for producing ethanol, the method comprising:

reacting the cellulolytic industrial yeast with a cellulose-based material to yield ethanol.

### EFFECTS OF THE INVENTION

According to the present invention, a foreign gene can be favorably introduced even into a yeast cell that does not have an auxotrophic marker. The method of the present invention can simultaneously provide an auxotrophic marker to a yeast cell and introduce a foreign gene into the yeast cell.

### BRIEF DESCRIPTION OF DRAWINGS

FIG. 1-1 is a schematic diagram showing the first half of the procedure of constructing a plasmid pBlue HU-BGL13 used for the marker recycling gene introduction for histidine gene (HIS3) disruption and β-glucosidase gene integration.

FIG. 1-2 is a schematic diagram showing the second half of the procedure of constructing a plasmid pBlue HU-BGL13 used for the marker recycling gene introduction for histidine gene (HIS3) disruption and β-glucosidase gene integration.

FIG. 2 is a schematic diagram showing the procedure of histidine gene (HIS3) disruption and β-glucosidase gene integration.

FIG. 3 shows an electrophoretogram showing the results of colony PCR to check whether the disruption of the HIS3 gene and the integration of the expression cassette for β-glucosidase cell surface displaying gene have occurred, together with a schematic diagram showing primers used in the colony PCR and regions to be amplified therewith.

FIG. 4 shows an electrophoretogram showing the results of colony PCR to check whether the URA3 marker is eliminated from the transformant obtained by selection by a 5-FOA medium, together with a schematic diagram showing primers used in the colony PCR and regions to be amplified therewith.

FIG. 5 is a graph showing the time course of the amounts of cellobiose and ethanol during fermentation for NBRC1440/HU-BGL13 and NBRC1440.

FIG. 6 is a schematic diagram of a plasmid pIHGP3-CBH AG that has a histidine gene (HIS3) marker and that is for integrating cellobiohydrolase (CBH2) gene so as to display on the surface.

FIG. 7 is a schematic diagram of a plasmid pGK406 EG that has a uracil gene (URA3) marker and that is for integrating endoglucanase (EGII) gene so as to display on the surface.

FIG. 8 is a graph showing the time course of the amount of ethanol produced from cellulose due to NBRC1440/HU-BGL13/pIHGP3-CBH/pGK406 EG, NBRC1440/HU-BGL13/pGK406 EG, and NBRC1440/HU-BGL13.

### MODES FOR CARRYING OUT THE INVENTION

### Construction and Use of Fragment for Gene Disruption and Gene Integration

In the present invention, a fragment for disrupting a target gene in a yeast cell, into which an expression cassette for a gene to be expressed in the yeast cell is inserted, is used. This fragment may also be called herein a “fragment for gene disruption and gene integration” for convenience. This fragment is used to disrupt a target gene for deletion and integrate a gene to be expressed for introduction. The term “gene disruption” refers to rendering a gene incapable of expressing in a host, and includes deletion of the gene from chromosome. This deletion may be achieved by the deletion of the region and the size necessary for a gene to be expressed and the entire sequence that constitutes the gene may not necessarily be deleted. The term “integration of gene” means that a gene is integrated into chromosome of host such that it can be expressed in the host. The term “introduction of gene” means that a gene is introduced into a cell to allow for its expression.

The fragment for gene disruption and gene integration contains two regions for homologous recombination, one of which is on each side of the expression cassette for a gene to be expressed. The fragment for gene disruption and gene integration may contain one or more expression cassette(s) for gene(s) to be expressed between the two regions for homologous recombination. The fragment may contain a linker if necessary. The regions for homologous recombination (sometimes, simply referred to as “homologous recombination regions”) refer to fragments that are homologous to regions located on both sides of a target gene in a yeast cell (that is, one is homologous to an upstream region (on the side of 5′ end) of the target gene and the other is homologous to a downstream region (on the side of 3′ end) of the target gene).

The “target gene” can be any gene the expression of which is not desirable in a yeast cell. In industrial yeasts that don't exhibit any auxotrophy useful for gene manipulation, the target gene may be a gene that controls the auxotrophy.

The expression cassette contains a gene to be expressed in a yeast cell, a promoter, and a terminator. The type of the gene to be expressed is not limited. For example, the gene may be a gene coding for a protein such as an enzyme. The promoter or the terminator may be that of the gene to be expressed or exogenous one may be added. As for the promoter and the terminator, promoters and terminators of GAPDH (glyceraldehyde 3′-phosphate dehydrogenase), PGK (phosphoglycerate kinase), GAP (glyceraldehyde 3′-phosphate), and like can be used, but the selection of promoter and terminator can be appropriately selected by those skilled in the art depending on the expression of the gene of interest. Optionally, a further factor that controls expression (for example, an enhancer) or the like may also be contained. The expression cassette may further contain a necessary functional sequence (e.g., of a cell surface-displaying technique, for a secretion signal, or the like as described in detail below) depending on the purpose of the expression of gene. The expression cassette may contain a linker as necessary.

The upstream-side one of the homologous recombination regions is homologous to a region upstream (on the side of 5′ end) of the target locus and can be arranged upstream (on the side of 5′ end) of the expression cassette (when more than one expression cassettes are present, one on the upstream side), and the downstream-side one of the homologous recombination regions is homologous to a region downstream (on the side of 3′ end) of the target locus and can be arranged downstream (on the side of 3′ end) of the expression cassette (when there are more than one expression cassettes, the cassette on the downstream side).

Herein, a region that is “homologous” to the base sequence means a region that has a sequence at least 90%, preferably at least 92%, more preferably at least 95%, still more preferably at least 97%, still more preferably at least 98%, still more preferably at least 99%, and most preferably at least 100% identical to the base sequence of the region referred to. Preferably, this “homologous region” is derived from the region referred to.

The length of the homologous recombination regions is not particularly limited. It is preferable that a region has a length suitable for allowing homologous recombination to occur. Therefore, the region may have a length of at least 40 base pairs.

The fragment for gene disruption and gene integration further contains a yeast selectable marker, thereby possibly facilitating the selection for a transformed yeast. Examples of the yeast selectable marker include markers that are usually used for selecting for transformed yeast cells, and often auxotrophy can be employed. If the target locus is a gene that controls an auxotrophy (i.e., a gene that enables a yeast to produce by itself a nutrient required for the growth of the yeast), the yeast selectable marker is a gene that controls a different auxotrophy from the target auxotrophy and is a counter selectable marker. The phrase “counter selectable” means allowing the defect or the disruption of the gene to be selected.

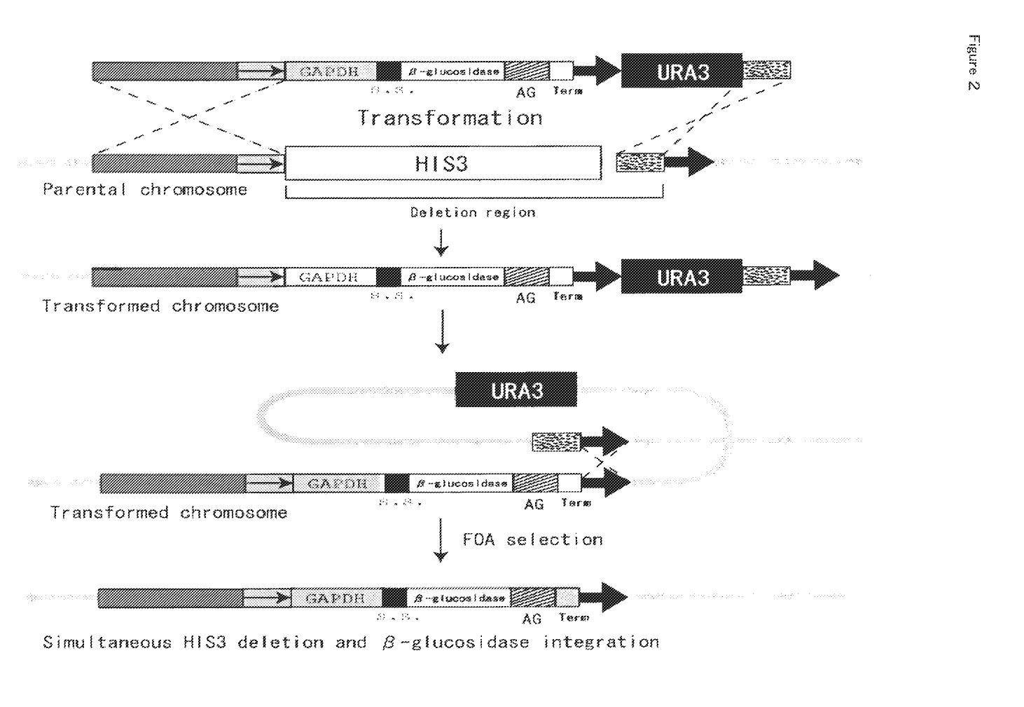

It is desirable that the yeast selectable marker is eliminated from a transformed yeast. For this purpose, a downstream sequence of the target locus can be employed. For easier understanding, description is provided in reference to FIG. 2. In the fragment for gene disruption and gene integration, the yeast selectable marker (“URA3” in the upper-most fragment in FIG. 2) takes the form of a cassette having, upstream (on the side of 5′ end) of the marker, a repeat region (sometimes simply referred to as the “repeat region” herein; the region indicated by a bold black arrow in the upper-most fragment in FIG. 2) that is homologous to a region (the region indicated by a bold black arrow in the “Parental chromosome” in FIG. 2) that is further downstream (on the side of 3′ end) of a region (the region indicated by a dotted strip in the “Parental chromosome” in FIG. 2) to which the downstream-side one of the homologous recombination regions (the region indicated by a dotted strip in the upper-most fragment of FIG. 2) is homologous. That is, the yeast selectable marker cassette contains a yeast selectable marker (“URA3” in the upper-most fragment in FIG. 2) and a repeat sequence (the region indicated by a bold black arrow in the upper-most fragment in FIG. 2) upstream (on the side of 5′ end) of the yeast selectable marker. Moreover, the yeast selectable marker cassette is arranged downstream (on the side of 3′ end) of the expression cassette and upstream (on the side of 5′ end) of the downstream-side one of the homologous recombination regions. In the fragment, the two sequences present in series downstream of the target locus can be present in the positions opposite to their original positions relative to the yeast selectable marker (the sequence originally downstream can be upstream of the marker and the sequence originally upstream can be downstream of the marker).

The repeat region may have any length that allows homologous recombination. Preferably, the length is about 40 bp or greater. It is preferable that the downstream-side one of the homologous recombination regions has such a length that the eventual elimination of the region and the yeast selectable marker is not interrupted. In the Examples, for the ease of operation, the repeat region and the downstream-side one of the homologous recombination regions each have a length of 40 bp, but the length is not limited thereto.

The fragment for gene disruption and gene integration can be incorporated into a vector. It is preferable for facilitating to obtain a DNA that the vector is a shuttle vector for use with Escherichia coli. It is more preferable that, for example, the vector has origin of replication (Ori) of 2 μm plasmid for yeast and origin of replication of ColE1 as well as a selectable marker for E. coli (a drug-resistant gene or the like) and a selectable marker for yeast (described above).

Transformation may be carried out according to a method well known to those skilled in the art. Specifically, examples include the lithium acetate method and the protoplast method. Any method may be used as long as the fragment itself is integrated into a host chromosome of a host through homologous recombination with the chromosome. For transformation, the fragment may be introduced into a cell in a form incorporated into a vector, such as a plasmid, or in the form of a linear fragment.

The transformation of a yeast cell by the fragment for gene disruption and gene integration results in homologous recombination between the fragment and the genome of the yeast cell using the homologous recombination regions, and thereby the target locus is disrupted and deleted, and the gene to be expressed and the yeast selectable marker are inserted with the repeat regions. Thus, a homologous region is arranged on each side of the yeast selectable marker and the downstream-side one of the homologous recombination regions (a repeat region and a region further downstream (on the side of 3′ end) of a region in the target locus to which the downstream-side one of the homologous recombination regions is homologous; both represented by the regions indicated by bold black arrows in the “Transformed chromosome” in FIG. 2). Furthermore, these homologous regions undergo homologous recombination and the yeast selectable marker therebetween is removed, leaving only the gene to be expressed on the chromosome. Once transformation is initiated, the target locus is disrupted and the gene to be expressed and the yeast selectable marker are first inserted due to a first homologous recombination, and the yeast selectable marker is then eliminated due to a second homologous recombination when culturing is continued.

According to the present invention, it is possible to provide an auxotrophy to a yeast cell and to introduce a gene to be expressed into the yeast cell. Therefore, the present invention may be advantageously used to introduce a foreign gene into a yeast that has originally no auxotrophy. According to the present invention, an auxotrophy, which is suitable as a marker for gene introduction, is provided even to a yeast that has no auxotrophy, thereby making it possible to select for a transformed cell into which a gene to be expressed is introduced. Examples of yeasts having no auxotrophy include industrial yeasts.

In order to provide an auxotrophy to and introduce a gene to be expressed into a yeast cell, any auxotrophy-controlling genes are used as the target locus. The “target auxotrophy-controlling gene” may be a gene that controls an auxotrophy not exhibited in the yeast cell to be transformed. Such an anxotrophy includes any auxotrophies applicable as a marker for selecting for a transformant. Examples of auxotrophies and genes that control such auxotrophies (which are also simply be called “auxotrophy-controlling genes” herein) include (corresponding auxotrophy-controlling genes are given in parentheses): uracil auxotrophy (for example, URA3, URA5), trypsin auxotrophy (for example, TRP1), leucine auxotrophy (for example, LEU2), histidine auxotrophy (for example, HIS3), and the like. The sequence information of such genes is available to those skilled in the art (for example, from the database of the Saccharomyces Genome Database http://www.yeastgenome.org/), and primers can be designed for the preparation of the fragment for gene disruption and gene integration using the sequence information of such genes.

Also, in order to facilitate the selection of a transformed yeast, an auxotrophic marker can be used as the yeast selectable marker. In this case, a gene that controls a different auxotrophy from the auxotrophy controlled by the targeted gene may be used. In order to use a gene that controls a different auxotrophy as a selectable marker for a transformed yeast, the yeast cell to be transformed needs to have this different auxotrophy. A yeast cell that has this different auxotrophy may be used as a transformation material or this different auxotrophy may be provided to a yeast prior to transformation. In the case of yeast having no auxotrophy such as industrial yeasts, this different auxotrophy is provided. For example, a counter selectable marker is desirable as the auxotrophic marker, and examples thereof include a gene for uracil auxotrophy (for example, URA3) and a gene for lysine auxotrophy (for example, LYS2). In consideration of the ease of the operation for providing an auxotrophy to a yeast and the applicability as a yeast selectable marker, URA3, which is a gene for uracil auxotrophy, can be preferably used for a different auxotrophy.

A marker for a different auxotrophy can be provided by disrupting the gene that controls the auxotrophy. In the case of providing uracil auxotrophy, for example, a normal ura3 gene of an industrial yeast can be replaced with an ura3⁻ fragment obtained from a uracil auxotrophic mutant (for example, a Saccharomyces cervisiae MT-8 strain) to disrupt the normal ura3 gene. In the case of a ura3 gene-disrupted strain, the presence/absence of a marker can be easily identified or selected by taking advantage of the fact that a ura3 gene-disrupted strain is able to grow in a medium containing 5-fluoroorotic acid (5-FOA) while a normal ura3 strain (wild-type yeast or usual industrial yeast) is not able to grow. In the case of a lys2 gene-disrupted strain, the presence/absence of a marker can be easily identified or selected by taking advantage of the fact that a ura3 gene-disrupted strain is able to grow in a medium containing α-aminoadipic acid while a normal lys2 strain (wild-type yeast or usual industrial yeast) is not able to grow. Methods for disrupting an auxotrophy-controlling gene and for selectively separating auxotrophy-controlling gene mutants may be used depending on the auxotrophy employed.

Where the target locus is a target auxotrophy-controlling gene and a gene for controlling a different auxotrophy is used as the yeast selectable marker, the phenotype of the different auxotrophy can be used to select for a transformed yeast. For example, in order to use a uracil auxotrophy-controlling gene (for example, URA3) as the yeast selectable marker, a yeast to be transformed is made uracil auxotrophic prior to transformation, and the expression of the uracil auxotrophy-controlling gene (i.e., a phenotype exhibiting no uracil auxotrophy) can be used for the selection for a transformed yeast from which the target auxotrophy-controlling gene is deleted and into which the gene to be expressed is introduced, and the selection using 5-FOA (i.e., the phenotype of exhibiting uracil auxotrophy) can be used for the confirmation of the recovery of the yeast selectable marker (uracil auxotrophy-controlling gene) (i.e., the elimination of the uracil auxotrophy-controlling gene from the transformed yeast), thereby, eventually, producing a yeast from which the target auxotrophy-controlling gene and the different-auxotrophy controlling gene are deleted to exhibit auxotrophies therefor and into which the gene to be expressed is introduced is produced.

According to the present invention, the auxotrophic marker provided to a yeast cell can be used for further gene manipulation. Therefore, into a transformed yeast into which the gene to be expressed has been introduced as prepared according to the present invention, a further gene can be introduced using the auxotrophic marker of the transformed yeast. For example, as described in the paragraph immediately above, where a uracil auxotrophy-controlling gene (for example, URA3) is used as a yeast selectable marker, uracil auxotrophy is included in the auxotrophy provided to the transformed yeast.

Therefore, a method for repetitively introducing genes into a yeast cell is provided according to the present invention. A yeast transformed with the fragment for gene disruption and gene integration (a yeast in which an auxotrophy-controlling gene is deleted and a gene to be expressed is introduced) may be further transformed. For such transformation, a vector containing an auxotrophy-controlling gene as deleted from the transformed yeast cell and an additional gene to be expressed can be used. The additional gene to be expressed may be the same as or different from the gene introduced into the yeast transformed with the fragment for gene disruption and gene integration. Moreover, the vector containing an auxotrophy-controlling gene as deleted from the transformed yeast cell and an additional gene to be expressed may contain two or more additional genes to be expressed (which may be the same or different).

It is desirable that the vector for expression contains, for the expression of the structural gene of an enzyme of interest, as it is called, a regulatory sequence, such as operator, promoter, terminator, or enhancer, which controls the expression of the gene. As for the promoter and the terminator, promoters and terminators of GAPDH (glyceraldehyde 3′-phosphate dehydrogenase), PGK (phosphoglycerate kinase), GAP (glyceraldehyde 3′-phosphate), and like can be used, but the selection of promoter and terminator can be appropriately selected by those skilled in the art depending on the expression of an enzyme gene of interest. The operator and the enhancer can be appropriately selected by those skilled in the art. For example, a plasmid containing GAPDH promoter and GAPDH terminator (for example, pYGA2270, pYE22m, pBG211, and the like), a plasmid containing PGK promoter and PGK terminator (for example, Yip-PGKp-YAP1 (TOYOBO) and the like), a plasmid containing GAP promoter and GAP terminator (for example, pYGA2270, pYE22m, pBG211, and the like), and the like may be used.

Examples of the vector include multi-copy vectors and chromosome integration vectors. It can be decided by those skilled in the art what gene is incorporated into what vector. Genes to be introduced may be incorporated into the same vector or each may be incorporated into different vectors.

The phrase “introduction of a gene construct into a yeast” means that a gene construct is introduced into a cell to allow for expression. As the method for introducing a gene construct, those known to those skilled in the art, including transformation, transduction, transfection, co-transfection, electroporation, and the like are used, and specific examples include the lithium acetate method, the protoplast method, and the like. The gene construct to be introduced may be incorporated into a chromosome in the form of a plasmid, or by insertion into the gene of a host, or through homologous recombination with the gene of a host.

A transformed yeast into which the gene construct has been introduced can be selected with a selectable marker (for example, an auxotrophic marker as mentioned above). Further confirmation can be made by measuring the activity of the expressed protein.

An industrial yeast from which an auxotrophy is lost following the transformation with a vector containing an auxotrophy-controlling gene as deleted from the transformed yeast and also containing an additional gene to be expressed, is subject again to the introduction of gene using the fragment for gene disruption and gene integration as mentioned above, allowing for the provision of an auxotrophic marker and the introduction of a gene to be expressed.

(Cellulolytic Industrial Yeast Expressing an Enzyme that can Cleave a β1,4-glycosidic Bond)

According to the present invention, it is possible to introduce a gene into an industrial yeast that is highly industrially applicable and that does not have an auxotrophic marker which is necessary for gene manipulation.

The term “industrial yeast” refers to any yeasts used conventionally in ethanol fermentation (for example, sake yeasts, shochu yeasts, wine yeasts, beer yeasts, baker's yeasts, and the like). Among industrial yeasts, sake yeasts are preferable in regard to high ethanol fermentability and high ethanol resistance and genetic stability. An “industrial yeast” has high ethanol resistance and preferably is viable at ethanol concentrations of 10% or greater. Moreover, it is preferable that it has acid resistance, heat resistance, and the like. More preferably, it may be flocculable. Examples of industrial yeast which has such properties include the Saccharomyces cerevisiae NBRC1440 strain (MATα, haploid yeast, heat resistant and acid resistant, flocculable) and the NBRC1445 strain (MATa, haploid yeast, heat resistant and acid resistant, not flocculable) both available from the National Institute of Technology and Evaluation.

Since the industrial yeast has extremely strong ethanol resistance, it is possible to apply it directly to ethanol fermentation after monosaccharide production. In particular, the industrial yeast is preferable because it is resistant to any stresses under culturing and shows stable cell proliferation even in industrial production where a precise control of culturing conditions is difficult, which may result in severe culturing conditions. Since industrial yeasts form polyploids, it is possible to integrate a plurality of gene constructs (expression vectors) into homologous chromosomes, and as a result, the amount of the protein of interest expressed is higher compared to the integration into laboratory yeasts, which are often haploids are integrated.

Industrial yeasts are often prototrophs and do not have an auxotrophic marker suitable for selecting for a transformant. According to the present invention, it is possible to provide a specific auxotrophic marker suitable for introducing a gene of interest to, and introduce the gene of interest into an industrial yeast, especially, into a yeast that does not have an auxotrophy and that is highly ethanol resistant (preferably that is viable at ethanol concentrations of 10% or greater). Examples of the auxotrophic marker provided by the fragment for gene disruption and gene integration include, but not limited to, uracil auxotrophy, trypsin auxotrophy, leucine auxotrophy, histidine auxotrophy, and the like, in view of the applicability in gene manipulation. As previously described, a normal ura3 gene of industrial yeast can be replaced with an ura3⁻ fragment obtained from an uracil auxotrophic mutant (for example, a Saccharomyces cerevisiae MT-8 strain) to provide uracil auxotrophy. Therefore, in consideration of the efficiency of gene manipulation, it is preferable to design the fragment so as to adopt as the target an auxotrophy other than uracil auxotrophy (for example, trypsin auxotrophy, leucine auxotrophy, histidine auxotrophy, or the like) and disrupt the gene thereof. An industrial yeast that has acquired uracil auxotrophy by the replacement of the normal ura3 gene of the industrial yeast with the ura3⁻ fragment of an uracil auxotrophic mutant is transformed with a fragment for gene disruption and gene integration designed to contain a uracil auxotrophy-controlling gene in the selectable enzyme marker cassette as mentioned above and designed to provide an auxotrophy (for example, trypsin auxotrophy, leucine auxotrophy, histidine auxotrophy, or the like) so as to obtain a transformed industrial yeast that has acquired uracil auxotrophy in addition to the target auxotrophy and into which the desired gene has been introduced.

Hereinbelow, the production of an expression cassette for an enzyme that can cleave a β1,4-glycosidic bond and that is highly industrially applicable to expression in industrial yeasts shall be described.

The “enzyme that can cleave a β1,4-glycosidic bond” herein is not particularly limited as long as it can cleave a β1,4-glycosidic bond. Typical examples may be cellulolytic enzymes, and preferable may be endo β1,4-glucanase (hereinafter simply called “endoglucanase”), cellobiohydrolase, glucan 1,4-βendoglucosidase, β-glucosidase, carboxymethylcellulase, or the like. Endoglucanase is an enzyme that is usually referred to as cellulase and it can cleave cellulose intramolecularly to generate glucose, cellobiose, and cello-oligosaccharide. Cellobiohydrolase can degrade cellulose from either the reducing end or the nonreducing thereof to liberate cellobiose. Glucan 1,4-βendoglucosidase can degrade cellulose from either the reducing end or the nonreducing end thereof to liberate glucose. β-glucosidase is an exo-hydrolase that liberates glucose units from the nonreducing end of cellulose. Carboxymethyl cellulase can degrade amorphous cellulose such as carboxymethylcellulose, which is a solubilized derivative by introducing a carboxymethyl group into some of the glucose residues of the cellulose chain.

The enzyme that can cleave a β1,4-glycosidic bond may be derived from any microorganism that produces a cellulolytic enzyme. Typical examples of cellulolytic enzyme-producing microorganisms include those belonging to the genus Aspergillus (for example, Aspergillus aculeatus, Aspergillus niger; and Aspergillus oryzae), the genus Trichoderma (for example, Trichoderma reesei), the genus Clostridium (for example, Clostridium thermocellum), the Cellulomonas (for example, Cellulomonas fimi and Cellulomonas uda), the genus Pseudomonas (for example, Pseudomonas fluorescence), and the like.

More specifically, as for cellulolytic enzymes for expression in industrial yeasts, β-glucosidase (especially BGL1) derived from Aspergillus aculeatus, endoglucanase (especially EGII) derived from Trichoderma reesei; and cellobiohydrolase (especially CBH2) derived from Trichoderma reesei may be used.

In order to degrade cellulose to produce glucose, a recombinant industrial yeast may be produced such that a combination of endoglucanase, cellobiohydrolase, and β-glucosidase can react with cellulose. According to the present invention, a recombinant industrial yeast can be produced so as to express only one of these enzymes in large amounts (to express a plurality of genes of the same enzymes) and/or to express a combination of two or more different enzymes. Therefore, in order to degrade cellulose to produce glucose, industrial yeasts each recombined to be able to express only one of these enzymes are combined or alternatively, industrial yeasts expressing two or more of these enzymes are combined, so that endoglucanase, cellobiohydrolase, and β-glucosidase can act on cellulose. Examples of combinations of two or more enzymes include (1) a combination of endoglucanase and β-glucosidase, (2) a combination of cellobiohydrolase and β-glucosidase, and (3) a combination of endoglucanase, cellobiohydrolase, and β-glucosidase. Such combinations allow cellulose to be efficiently degraded to produce glucose.

The gene of an enzyme to be expressed may be obtained from a microorganism that produces this enzyme according to PCR or hybridization using primers or a probe designed based on known sequence information.

Examples of methods for producing a microorganism that displays an enzyme on the cell surface include, although they are not limited to, (a) displaying an enzyme on cell surface via the GPI anchor of a cell surface-localized protein, (b) displaying an enzyme on cell surface via the sugar chain-bound protein domain of a cell surface-localized protein, and (c) displaying an enzyme on the cell surface via a periplasmic protein (another receptor molecule or target receptor molecule). Relevant techniques for cell surface engineering are described in, for example, Patent Documents 1 to 3 (especially, Patent Document 3).

Examples of usable cell surface-localized proteins include α- or a-agglutinin, which is a yeast flocculation protein (for use as a GPI anchor); Flo1 proteins (Flo1 proteins can be used as GPI anchors with modification of the amino acid length on the N-terminal; for example, Flo42, Flo102, Flo146, Flo318, Flo428, and the like; Non-Patent Document 4: Note that Flo1326 refers to the full-length Flo1 protein); Ho proteins (there are no GPI anchor functions and flocculability is used, Floshort or Flolong; Non-Patent Document 5); invertase, which is a periplasm-localized protein (no GPI anchor is used); and the like.

First, (a) use of GPI anchor shall be described. The gene coding for a protein localized on cell surface by a GPI anchor has, in order from the N-terminal, a gene coding for a secretion signal sequence, a gene coding for a cell surface-localized protein (a sugar chain-bound protein domain), and a gene coding for a GPI anchor attachment recognition signal sequence. The cell surface-localized protein (sugar chain-bound protein) is expressed from this gene in the cell and directed outside the cell membrane by a secretion signal, and then its GPI anchor attachment recognition signal sequence binds to the GPI anchor of the cell membrane via a specifically truncated C-terminal portion to immobilize the protein on the cell membrane. Subsequently, the protein is cleaved near the root of the GPI anchor by PI-PLC, and integrated into the cell wall and immobilized on the surface of the cell, resulting in display of the protein on cell surface.

Here, the GPI anchor refers to a glycolipid having a basic structure of ethanolamine-phosphate-6-mannose-α1-2-mannose-α1-6-mannose-α1-4-glucosamine-α1-6-i nositol-phospholipid, called glycosyl phosphatidylinositol (GPI), and PI-PLC refers to phosphatidylinositol-dependent phospholipase C.

The GPI anchor attachment recognition signal sequence can be recognized upon the binding the GPI anchor to the cell surface-localized protein and is usually located at or near the C-terminal of the cell surface-localized protein. As for the GPI anchor attachment signal sequence, for example, the sequence of the C-terminal portion of α-agglutinin of yeast is preferably used. Since the GPI anchor adhesion recognition signal sequence is contained on the C-terminal region of the sequence of 320 amino acids from the C-terminal of the α-agglutinin, a DNA sequence coding for the sequence of 320 amino acids from the C-terminal is particularly useful as a gene for use in the aforementioned method.

Therefore, for example, in a sequence containing a DNA coding for a secretion signal sequence-a structural gene coding for a cell surface-localized protein-a DNA sequence coding for a GPI anchor attachment recognition signal, the entire or a part of the sequence of the structural gene coding for cell surface-localized protein can be replaced with a DNA sequence coding for the enzyme of interest so as to obtain a recombinant DNA for displaying the enzyme of interest on the cell surface via the GPI anchor. Where the cell surface-localized protein is α-agglutinin, it is preferable to introduce a DNA coding for the enzyme of interest such that the sequence coding for the sequence of 320 amino acids from the C-terminal of the α-agglutinin is retained. The enzyme which is displayed on the cell surface by introducing into and expressing such a DNA in a yeast is immobilized on the surface via the C-terminal.

Next, (b) use of sugar chain-bound protein domain shall be described. Where the cell surface-localized protein is a sugar chain-bound protein, the sugar chain-bound protein domain has a plurality of sugar chains and the sugar chains interact or are entangled with sugar chains present in the cell wall, thereby allowing the protein to left on the cell surface. Examples include sugar chain-binding sites of lectin, lectin-like proteins, and the like. Typical examples include the flocculation functional domain of a GPI anchor protein and the flocculation functional domain of a FLO protein. The flocculation functional domain of a GPI anchor protein refers to a domain that is located on the side of N-terminal relative to the GPI anchoring domain, has a plurality of sugar chains, and is thought to be involved in flocculation.

The linkage of the sugar chain-bound protein domain (or the flocculation functional domain) of a cell-surface localized protein with the enzyme of interest allows the enzyme to be displayed on the cell surface. Depending on the type of the enzyme of interest, the enzyme may be bound (1) on the side of N-terminal or (2) on the side of C-terminal of the sugar chain-bound protein domain (or the flocculation functional domain) of a cell surface-localized protein, or the same or different enzymes may be bound (3) on both sides of the N-terminal and C-terminal. In the present invention, (1) a DNA coding for a secretion signal sequence-a gene coding for a target enzyme-a structural gene coding for the sugar chain-bound protein domain (or the flocculation functional domain) of a cell surface-localized protein; (2) a DNA coding for a secretion signal sequence-a structural gene coding for the sugar chain-bound protein domain (or the flocculation functional domain) of a cell surface-localized protein-a gene coding for a target enzyme; or (3) a DNA coding for a secretion signal sequence-a first gene coding for a target enzyme-a structural gene coding for the sugar chain-bound protein domain (or the flocculation functional domain) of a cell surface-localized protein-a second gene coding for the target enzyme (wherein the first gene and the second gene may be the same or different) can be prepared to obtain a recombinant DNA for displaying the enzyme of interest on the cell surface. Where the flocculation functional domain is used, the DNA sequence coding for a GPI anchor attachment recognition signal sequence may be partially present or may not be present in the recombinant DNA since the GPI anchor is not involved in cell surface display. The use of the flocculation functional domain is very advantageous in that: the enzyme can be displayed in more suitable length on the cell surface because the length of the domain can be easily modified (for example, any of Floshort and Flolong can be adopted); and it can be linked on either side of the N-terminal or C-terminal of the enzyme.

Next, (c) use of periplasmic protein (another receptor molecule or target receptor molecule) shall be described. This method is based on the fact that the enzyme of interest can be expressed on the cell surface as a fused protein with the periplasmic protein. An example of a periplasm-free protein may be invertase (Suc2 protein). The enzyme of interest may be appropriately fused on the side of N-terminal or C-terminal depending on the periplasmic protein.

The method for secretary expression of enzyme out of cell for yeast is well known to those skilled in the art. A recombinant DNA in which the structural gene of the enzyme of interest is linked to a DNA coding for the secretion signal sequence may be prepared and introduced into a yeast.

Naturally, the method for expression of enzyme in cell for yeast is also well known to those skilled in the art. In this case, a recombinant DNA in which the structural gene of interest is linked without the cell surface displaying technique or the secretion signal as mentioned above may be prepared and introduced into a yeast.

The synthesis and the linkage of DNAs including various sequences as described above may be performed using techniques commonly used by those skilled in the art. For example, the binding of the structural gene of the enzyme of interest with the secretion signal sequence can be carried out by way of site specific mutation, thereby allowing accurate cleavage of the secretion signal sequence and active expression of the enzyme.

It is desirable that the expression cassette contains, as it is called, a regulatory sequence, such as operator, promoter, terminator, or enhancer, that controls the expression of the gene. As for the promoter and the terminator, promoters and terminators of GAPDH (glyceraldehyde 3′-phosphate dehydrogenase), PGK (phosphoglycerate kinase), GAP (glyceraldehyde 3′-phosphate), and like can be used, but the selection of promoter and terminator can be appropriately selected by those skilled in the art depending on the expression of the enzyme gene of interest. The operator and the enhancer can be appropriately selected by those skilled in the art.

The transformation of an industrial yeast with the fragment for gene disruption and gene integration which contains the expression cassette as mentioned above is as described above. The expression cassette can be designed such that the enzyme can be displayed on cell surface so as to obtain an industrial yeast that displays such enzymes on the surface.

The industrial yeast into which the gene to be expressed has been introduced by the integration of the expression cassette as mentioned above may be selected with a yeast selectable marker (for example, an auxotrophic marker mentioned above) as described above, and confirmed by determining the activity of the expressed protein. The immobilization of protein on cell surface may be confirmed by immunological antibody method using an anti-protein antibody and an FITC-labeled anti-IgG antibody.

According to the present invention, an industrial yeast that expresses endoglucanase, cellobiohydrolase, and β-glucosidase in combination can be prepared. For example, the fragment for gene disruption and gene integration is designed such that it contains an expression cassette for β-glucosidase, allows for providing histidine auxotrophy, and contains a yeast selectable marker cassette for a uracil auxotrophy controlling gene (for example, URA3). An industrial yeast is provided beforehand with uracil auxotrophy. Subsequently, the industrial yeast which uracil auxotrophy has been previously provided is transformed with the aforementioned fragment. The selection for transformed industrial yeast may be carried out as described above. By this stage, the industrial yeast is provided with uracil auxotrophy and histidine auxotrophy and has β-glucosidase gene introduced. Next, a vector for the expression of endoglucanase containing a uracil auxotrophy selectable marker and a vector for the expression of cellobiohydrolase containing a histidine auxotrophy selectable marker (the markers and the expression genes may be in opposite combinations) are constructed, and the transformed yeast is further transformed with these expression vectors, eventually yielding an industrial yeast that expresses endoglucanase, cellobiohydrolase, and β-glucosidase in combination. Alternatively, the transformed yeast to which uracil and histidine markers have been provided can also be further transformed with a vector constructed such that it contains a uracil auxotrophy selectable marker or a histidine auxotrophy selectable marker and allows for expressing endoglucanase and cellobiohydrolase. The expression cassettes for endoglucanase, cellobiohydrolase, and β-glucosidase can be constructed such that endoglucanase, cellobiohydrolase, and β-glucosidase are displayed on cell surface or secreted. As with the histidine auxotrophy selectable marker, a trypsin auxotrophy selectable marker (for example, TRP1) or a leucine auxotrophy selectable marker (for example, LEU2) can also be used in providing a marker to an industrial yeast as well as in preparing a recombinant of the cellulase-expressed industrial yeast as mentioned above.

(Production of ethanol)

The present invention provides a method for producing ethanol using a cellulolytic industrial yeast, which expresses, or preferably displays on the surface, the enzyme that can cleave a β1,4-glycosidic bond. This method includes the step of reacting a cellulolytic industrial yeast recombined to express on the surface an enzyme that can cleave a β1,4-glycosidic bond with a cellulose-base material to yield ethanol. Using the cellulolytic industrial yeast, glucose is obtained from cellulose present in the cellulose-based material and ethanol is generated using the resulting glucose.

The term “cellulose-based material” as used herein refers to any material, product, and composition containing cellulose. The term “cellulose” refers to a fibrous polymer in which glucopyranoses are connected by a β1,4-glycosidic bond and includes derivatives and salts thereof as well as those that have a degree of polymerization reduced by decomposition. For example, carboxymethylcellulose (CMC) in which cellulose is carboxymethylated, phosphoric acid-swollen cellulose, and the like are included. Crystalline cellulose (for example, Avicel) is also usable. Cellulose may be present also in industrial waste (for example, paper waste generated in paper production or recycling, and the xylem of trees and the leaves, stems, bark, and the like (especially, non-edible portions) of herbaceous plants that are not agriculturally harvested or are disposed of in food production), and such waste is encompassed within the term “cellulose-based material.”

The xylem of trees and the leaves, stems and bark of herbaceous plants may contain plant cell wall components, one of the principal components of which is cellulose. Plant cell walls usually contain, in addition to cellulose, hemicellulose and lignin as their components. Depending on the plant species (especially, whether woody or herbaceous), the extent of plant growth, or the like, the contents of such components may vary, but a plant of any species at any growth stage may be used as long as it contains cellulose.

Therefore, cellulose-based materials also include any material, waste, and product containing the plant cell wall components as mentioned above. Insoluble dietary fiber is also encompassed within the term “plant cell wall component-containing materials.” Cellulose-based materials include, in addition to the xylem of trees and the leaves, stems and bark of herbaceous plants as mentioned above, products processed from such portions (for example, corn fiber), but use of waste to be disposed of from is preferable in the present invention in view of reuse.

Cellulose-based materials (especially, cellulose fiber in waste) may be treated using the non-catalytic hydrothermal method, for example, according to Patent Document 3, for example, such that cellulose units or oligosaccharides are formed in suitable length or such that the crosslinks between fiber chains (for example, inter-cellulose crosslinks) are uncoupled so as to allow a cellulolytic enzyme to readily acts thereon.

The xylem of trees and the leaves, stems and bark of herbaceous plants may be used in the method of the present invention, preferably after removing non-glycosylated portions such as lignin.

In the method described above, the cellulolytic industrial yeast may be added such that cellulose is degraded to produce glucose. An industrial yeast may be recombinantly prepared such that a combination of endoglucanase, cellobiohydrolase, and β-glucosidase can react with cellulose. According to the present invention, a recombinant industrial yeast can be produced so as to express in large amounts one of these enzymes and/or to express a combination of two or more different enzymes. Therefore, in order to degrade cellulose to produce glucose, industrial yeasts each recombined to be able to express only one of these enzymes are combined, or alternatively, industrial yeasts expressing two or more of these enzymes are combined, so that endoglucanase, cellobiohydrolase, and β-glucosidase can act on cellulose. Examples of combinations of two or more enzymes include (1) a combination of endoglucanase and β-glucosidase, (2) a combination of cellobiohydrolase and β-glucosidase, and (3) a combination of endoglucanase, cellobiohydrolase, and β-glucosidase. Such combinations allow cellulose to be efficiently degraded to produce glucose.

According to the present invention, an industrial yeast recombined to express on the cell surface one of β-glucosidase (especially BGL1derived from Aspergillus aculeatus), endoglucanase (especially EGII derived from Trichoderma reesei), and cellobiohydrolase (especially CBH2 derived from Trichoderma reesei) or two or more of these is provided. Such yeasts are preferable as yeasts for use in ethanol production.

In order to produce ethanol from cellulose-based materials, xylose utilization can also be utilized in addition to cellulolytic properties. Xylose can be obtained from the enzymolysis of hemicellulose contained in cell wall components one of the principal components of which is cellulose. Xylose from hemicellulose can also be used for ethanol fermentation by separately producing an industrial yeast that expresses a xylose utilization gene and/or a gene coding for a xylanolytic enzyme or by expressing also such a gene in a cellulolytic industrial yeast for xylose utilization. It is preferable that a hemicellulose-degrading enzyme (for example, a xylanolytic enzyme) is displayed on the cell surface of an industrial yeast. Examples of xylanolytic enzymes include xylanase (especially XYLII derived from Trichoderma reesei) and β-xylosidase (XylA derived from Aspergillus oryzae). Xylose utilization genes include genes for xylose-metabolising enzymes and examples of which include xylose reductase (XR) gene and xylitol dehydrogenase (XDH) gene (both derived from Pichia stipitis) and xylulokinase (XK) gene (derived from Saccharomyces cerevisiae).

For example, in order to prepare an industrial yeast that has both xylanolytic (hemicellulose-degrading) and xylose-metabolizing properties, an industrial yeast may be recombinantly produced to express xylanase (especially XYLII derived from Trichoderma reesei (INSD Accession No. X69574;S51975)) and β-xylosidase (XylA derived from Aspergillus oryzae (INSD Accession No. AB013851)) on the cell surface and express a xylose utilization gene (especially xylose reductase (XR) gene XYL1 derived from Pichia stipitis (INSD Accession No. X59465), xylitol dehydrogenase (XDH) gene XYL2 derived from Pichia stipitis (INSD Accession No. X55392), and xylulokinase (XK) gene XKS1 derived from Saccharomyces cerevisiae (INSD Accession No. X82408). For this purpose, the gene introduction using the fragment for gene disruption and gene integration as mentioned above may be used.

The recombinant industrial yeast used in the method of the present invention is preferably immobilized on a carrier, thereby making reuse possible.

The carrier and the method for immobilizing the recombinant industrial yeast may be a carrier and a method that are usually used by those skilled in the art. Examples include carrier binding, entrapment, crosslinking, and the like.

A porous material is preferably used as a carrier for immobilizing the recombinant industrial yeast. For example, preferable are polyvinyl alcohol, polyurethane foam, polystyrene foam, polyacrylamide, polyvinyl formal porous resin, silicone foam, and like foam and resin. The pore size of a porous material may be selected in consideration of the microorganism to be used and the size thereof. In the case of an industrial yeast, the size is preferably 50 to 1000 μm.

The carrier may have any shape. In view of the strength of the carrier, culturing efficiency, and the like, the shape is preferably spherical or cubic. The size may be selected according to the microorganism to be used, and it is generally preferable that the diameter is 2 to 50 mm where the carrier is spherical and one side has a length of 2 to 50 mm where the carrier is cubic.

The recombinant industrial yeast may be increased in number by culturing under aerobic conditions before the yeast is applied to fermentation for use in the method of the present invention. The medium may be a selective medium or a nonselective medium. The pH of the medium during culturing is preferably about 4.0 to about 6.0 and most preferably about 5.0. The concentration of dissolved oxygen in the medium during aerobic culturing is about 0.5 to about 6 ppm, more preferably about 1 to about 4 ppm, and most preferably about 2.0 ppm. The temperature during culturing may be about 20 to about 45° C., preferably about 25 to about 35° C., and most preferably about 30° C. It is preferable to culture until the cell concentration reaches 10 g/l or greater and the culturing time may be about 20 to about 50 hours.

In the method of the present invention, a cellulolytic industrial yeast and a cellulose-based material substrate are used for fermentation under such conditions that ethanol fermentation is usually carried out, so as to produce ethanol. A xylanolytic (hemicellulose-degrading) and/or xylose-metabolizing industrial yeast may be added during the fermentation. The cellulolytic industrial yeast may further possess xylanolytic (hemicellulose degrading) and/or xylose metabolizing properties. Examples of the mode of fermentation include a batch process, a feed-batch process, a repetitive batch process, a continuous process, and the like, and any of such processes may be selected. The temperature during fermentation may be about 30 to 35° C.

Since the fermentation conditions may change during the course of fermentation, it is preferable to control the conditions to be within a specific range. The time course of fermentation may be monitored by any means commonly used by those skilled in the art, such as, gas chromatography, HPLC, or a like.

In addition, a cellulase enzyme may be added to promote the ethanol production resulting from the saccharification of cellulose and the fermentation by the recombinant industrial yeast. A commercially available cellulase enzyme is usable.

After the completion of fermentation, the ethanol-containing medium is removed from the fermenter and ethanol is isolated by separation commonly used by those skilled in the art, such as centrifugation or distillation.

The present invention shall be described below by way of examples but is not limited by the examples.

### EXAMPLES

### Materials

A Saccharomyces cerevisiae NBRC1440 (MATα) strain was obtained from the National Institute of Technology and Evaluation, and used as a host for transformation, chromosome DNA preparation, and ethanol fermentation from β-glucan (cellobiose) or phosphoric acid-swollen cellulose (amorphous cellulose).

A Saccharomyces cerevisiae MT8-1 (MATa ade his3 leu2 trp1 ura3) strain was used as a template for URA3 mutation amplification (Non-Patent Document 6).

An E. coli DH5α strain was used for the construction and amplification of plasmids. The E. coli strain was grown in Luria-Bertani medium (10 g/L of tryptone, 5 g/L of yeast extract, and 5 g/L of sodium chloride) containing 100 mg/mL of ampicillin.

Yeast cells were aerobically cultured at 30° C. in synthetic dextrose (SD) medium (containing 6.7 g/L of a yeast nitrogen base without amino acids [manufactured by Difco], with appropriate supplements; to which 20 g/L of glucose was added as the sole carbon source) and in yeast extract-polypeptone-dextrose (YPD) medium (containing 10 g/L of a yeast extract, 20 g/L of polypeptone, and 20 g/L of glucose). Following fermentation, the yeast was anaerobically inoculated at 30° C. into yeast extract-polypeptone-cellobiose (YPCellobiose) medium (containing 10 g/L of yeast extract, 20 g/L of polypeptone, and 50 g/L of cellobiose).

The preparation of a 5-fluoroorotic acid (FOA) medium was as in the following manner. A uracil drop-out synthetic dextrose (SD) medium (Non-Patent Document 7) to which 50 mg/L of uracil acid and 2% (w/v) agar had been added was autoclaved and kept at 65° C. FOA was dissolved in dimethyl sulfoxide (DMSO) to a concentration of 100 mg/mL, and the resultant was added to the autoclaved medium at about 65° C. to the final FOA concentration of 1 mg/mL.

(PCR)

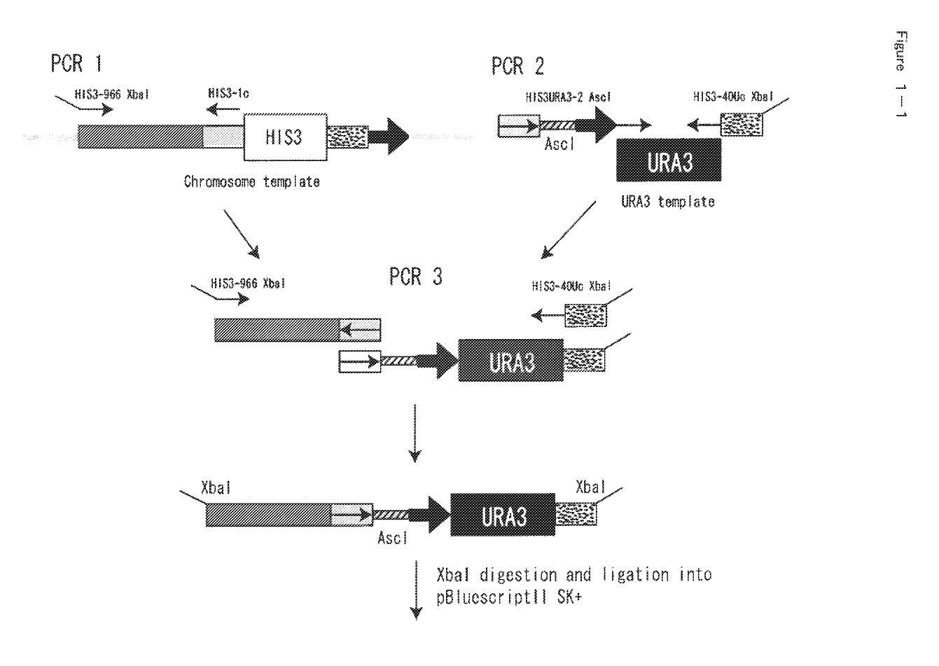

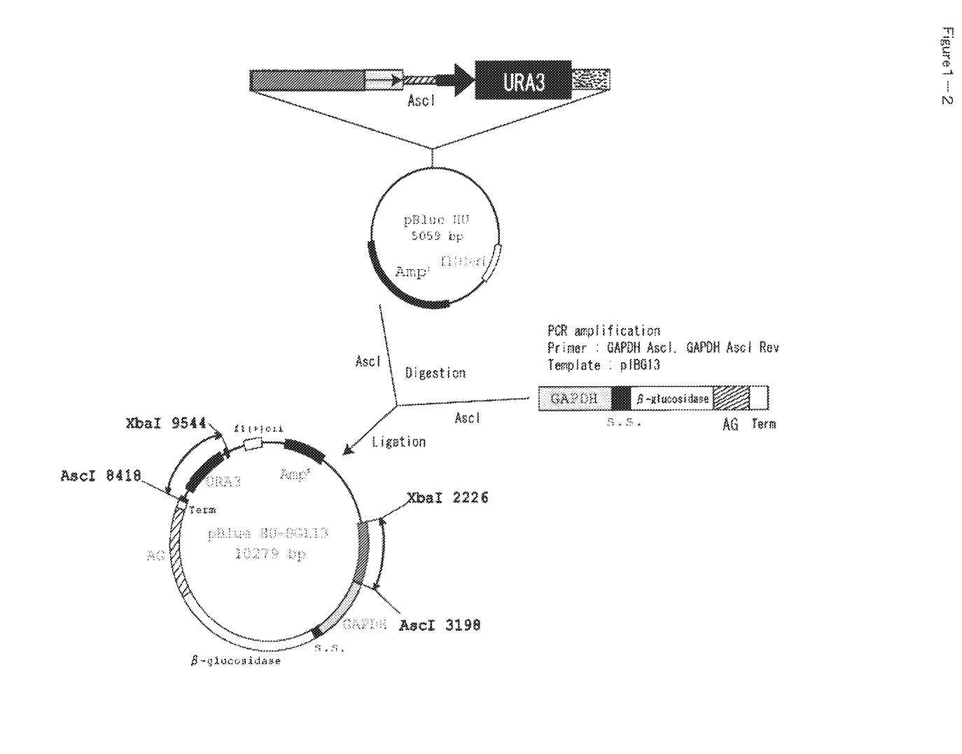

All PCR amplifications were performed using KOD-Plus-DNA polymerase (manufactured by Toyobo Co., Ltd.). The following primers were used: HIS3-966 XbaI (SEQ ID No. 1: the base sequence of bases 1 to 8 corresponds to an XbaI restriction enzyme site); HIS3-1c (SEQ ID No. 2: the base sequence of bases 1 to 24 corresponds to an overlapping sequence for fusion PCR); HIS3URA3-2 AscI (SEQ ID No. 3: the base sequence of bases 1 to 24 corresponds to an overlapping sequence for fusion PCR, the base sequence of bases 25 to 32 corresponds to the AscI restriction enzyme site, the base sequence of bases 33 to 72 corresponds to a repeat sequence (40 bp; the region indicated by a bold black arrow in FIG. 1) copied from an HIS3 gene locus, and the base sequence of bases 73 to 89 correspond to an annealing sequence for URA3 amplification); HIS3-40Uc XbaI (SEQ ID No. 4: the base sequence of bases 1 to 8 corresponds to the XbaI restriction enzyme site, the base sequence of bases 9 to 48 corresponds to a short sequence (40 bp; the region indicated by a dotted strip in FIG. 1) copied from the HIS3 locus, and the base sequence of bases 49 to 68 corresponds to an annealing sequence for URA3 amplification); HIS3-239c (SEQ ID No. 5; designed from a sequence in a region upstream (on the side of 5′ end) of the HIS3 gene); HIS3-124 (SEQ ID No. 6; designed from a sequence in a region downstream (on the side of 3′ end) of the HIS3 gene); HIS3 β-Glu check (SEQ ID No. 7; designed from the sequence of β-glucosidase gene); HIS3 β-Glu check2 (SEQ ID No. 8; designed from the sequence of β-glucosidase gene); a primer pair for preparation of the expression cassette for β-glucosidase (BGL1) surface display, GAPDH AscI (SEQ ID No. 9: bases 2 to 10 of the base sequence correspond to the AscI restriction enzyme site) and GAPDH AscI Rev (SEQ ID No. 10: the base sequence of bases 2 to 10 corresponds to the AscI restriction enzyme site); a primer pair for acquisition of a BGL1 gene fragment, bgl1 primer 1 (SEQ ID No. 11) and bgl1 primer 2 (SEQ ID No. 12); a primer pair for acquisition of a ΔURA3 fragment, URA3 delete (SEQ ID No. 13) and URA3 Δr (SEQ ID No. 14); a primer pair for preparation of the expression cassette for cellobiohydrolase (CBH2) surface display, R.ory s.s. XmaI (SEQ ID No. 15) and CBH XbaI Rev (SEQ ID No. 16); a primer pair for acquisition of a fragment containing GAPDH promoter, a multi-cloning site, and GAPDH terminator for preparation of pIHGP3, XYL2c-XhoI (F) (SEQ ID No. 17) and XYL2c-NotI (R) (SEQ ID No. 18); a primer pair for preparation of the expression cassette for endoglucanase (EG2) surface display, EG NheI Fw (SEQ ID No. 19) and EG XmaI Rev (SEQ ID No. 20); a primer pair for acquisition of PGK promoter for preparation of pGK406 (SEQ ID Nos. 21 and 22); a primer pair for acquisition of PGK terminator for preparation of pGK406 (SEQ ID Nos. 23 and 24); and a primer pair for acquisition of a multi-cloning site for preparation of pGK406 (SEQ ID Nos. 25 and 26).

(Colony PCR)

Colony PCR was performed as described in Non-Patent Document 8. PCR amplification was initiated at 94° C. for 5 minutes, then carried out using 30 cycles of 94° C. for 20 seconds, 50° C. for 30 seconds, and 68° C. for 2 minutes.

(Yeast Transformation)

Yeast transformation was carried out by lithium acetate method using YEAST MAKER yeast transformation system (manufactured by Clontech Laboratories, Palo Alto, Calif., USA).

### Example 1

### Construction of Plasmid Containing Fragment for Gene Disruption and Gene Integration

FIG. 1 shows a scheme for the construction of a plasmid pBlue HU-BGL13 used for the marker recycle gene introduction of histidine gene (HIS3) disruption and β-glucosidase gene integration (FIG. 1-1 shows the production through PCR of the HIS3-deleted fragment in the first half of the plasmid pBlue HU-BGL13 construction and FIG. 1-2 shows the ligation of the base plasmid with the HIS3-deleted fragment and the β-glucosidase expression cassette in the second half).

Fusion PCR was performed for the production through PCR of the HIS3-deleted fragment in the following manner.

PCR1, HIS3-966 XbaI (SEQ ID No. 1; Forward) and HIS3-1c (SEQ ID No. 2; Reverse) primers were used to amplify the HIS3 upstream sequence with the chromosome DNA of the Saccharomyces cerevisiae NBRC1440 strain as a template;

PCR2, HIS3URA3-2 AscI (SEQ ID No. 3; Forward) and HIS3-40Uc XbaI (SEQ ID No. 4; Reverse) primers were used for URA3 amplification using pRS406 plasmid (manufactured by Stratagene) as a template;

PCR3, HIS3-966 XbaI (SEQ ID No. 1; Forward) and HIS3-40Uc XbaI (SEQ ID No. 4; Reverse) primers were used to amplify about 2.1 kb of fusion fragment by mixing the PCR1 and PCR2 products as templates.

The resultant fragment (FIG. 1-1) contains a short region (40 bp region: the region indicated by a dotted strip downstream of HIS3 in FIG. 1-1) immediately downstream (on the side of 3′ end) of HIS3 in the chromosome DNA of Saccharomyces cerevisiae NBRC1440 and a short region (40 bp region: the region indicated by a bold black arrow in FIG. 1-1) immediately further downstream (on the side of 3′ end) thereof one on each side of the URA3 marker (the region indicated by a bold black arrow on the upstream side and the region indicated by a dotted strip on the downstream side) and further contains a region immediately upstream (on the side of 5′ end) of HIS3.

The resultant fusion fragment was digested with XbaI and introduced into the XbaI site of pBluescript II SK+ (manufactured by Takara Bio Inc.). The resultant plasmid was designated pBlue HU (FIG. 1-2).

Meanwhile, the β-glucosidase expression cassette was prepared in the following manner. First, the plasmid for displaying Aspergillus aculeatus-derived β-glucosidase (BGL1) on the cell surface, pIBG13, was constructed in the following manner. A 2.5 kbp NcoI-XhoI DNA fragment coding for Aspergillus aculeatus-derived β-glucosidase 1 (BGL1) gene was prepared through PCR with a primer pair of bgl1 primer 1 (SEQ ID No. 11; Forward) and bgl1 primer 2 (SEQ ID No. 12; Reverse) using a plasmid pBG211 (donated by Kyoto University) as a template. This DNA fragment was digested with NcoI and XhoI and inserted into the NcoI-XhoI site of a cell surface expression plasmid, pIHCS (Non-Patent Document 10) which contains a gene coding for the secretory signal sequence of Rhizopus oryzae-derived glucoamylase gene and the 3′ half of α-agglutinin gene (Non-Patent Document 9). The resultant plasmid was designated pIBG13.

Next, GAPDH AscI (SEQ ID No. 8; Forward) and GAPDH AscI Rev (SEQ ID No. 9; Reverse) primers were used to perform PCR amplification using pIBG13 as a template. Accordingly, a fragment (β-glucosidase expression cassette) containing Saccharomyces cerevisiae-derived glyceraldehyde-3-phosphate dehydrogenase (GAPDH) promoter, the secretory signal sequence (s.s.) of Rhizopus oryzae-derived glucoamylase gene, Aspergillus aculeatus-derived β-glucosidase gene, the 3′ half of the region coding for 320 amino acids of α-agglutinin (AG), 446 bp of 3′-flanking portion, and Saccharomyces cerevisiae-derived GAPDH terminator was obtained (FIG. 1-2).

The β-glucosidase expression cassette was digested with AscI, and introduced into the AscI site of pBlue HU. The resultant plasmid was designated pBlue HU-BGL13 (FIG. 1-2). In FIG. 1-2, the portion indicated by an arrow in pBlue HU-BGL13 is the marker recycle site for the HIS3 gene disruption.

### Example 2

### Provision of URA3 Marker to Industrial Yeast

The above-described laboratory yeast Saccharomyces cerevisiae MT8-1 strain is a uracil auxotrophic (URA3⁻) mutant. The ΔURA3 fragment was cloned from the MT8-1 strain genome through PCR amplification using a primer pair of URA3 delete (SEQ ID No. 13; Forward) and URA3 Δr (SEQ ID No. 14; Reverse). The fragment was transformed into the Saccharomyces cerevisiae NBRC1440 (MATα) strain, and the transformed strain was selected by the 5-FOA medium as described above.

### Example 3

### Preparation of Histidine Gene-Deleted and β-Glucosidase Gene-Integrated Industrial Yeast

FIG. 2 shows a scheme for histidine gene (HIS3) disruption and β-glucosidase gene integration. In FIG. 2, genetic sequences indicated by abbreviations and strips are the same as those in FIGS. 1-1 and 1-2.

The pBlue HU-BGL13 obtained according to Example 1 contains the construct inserted into the fragment for HIS3 gene disruption that contains a counter selectable URA3 marker as well as homologous targeting sequences which are located upstream and downstream of the marker, with a repeat sequence (the region indicated by a bold black arrow in the fragment) copied from the adjust region (the region indicated by the bold black arrow in the parental chromosome) of the targeted HIS3 locus being arranged downstream (on the side of 3′ end) of the expression cassette for β-glucosidase cell surface display (the uppermost schematic diagram in FIG. 2).

The plasmid pBlue HU-BGL13 was processed into a linear form using the restriction enzyme XbaI, and was transformed into the Saccharomyces cerevisiae NBRC1440 strain with which the URA3 marker had been provided (i.e., having uracil auxotrophy) as prepared according to Example 2, and a strain that had no uracil auxotrophy was selected on a uracil drop-out plate (a uracil-free medium). Integration of this construct into the industrial yeast NBRC1440 chromosome simultaneously generates the HIS3 gene disruption and the integration into the chromosome of the expression cassette for β-glucosidase cell surface displaying gene, the URA3 marker, and the repeat sequence arranged on both sides thereof (the schematic diagram shown immediately below the first downward-pointing arrow from the top in FIG. 2).

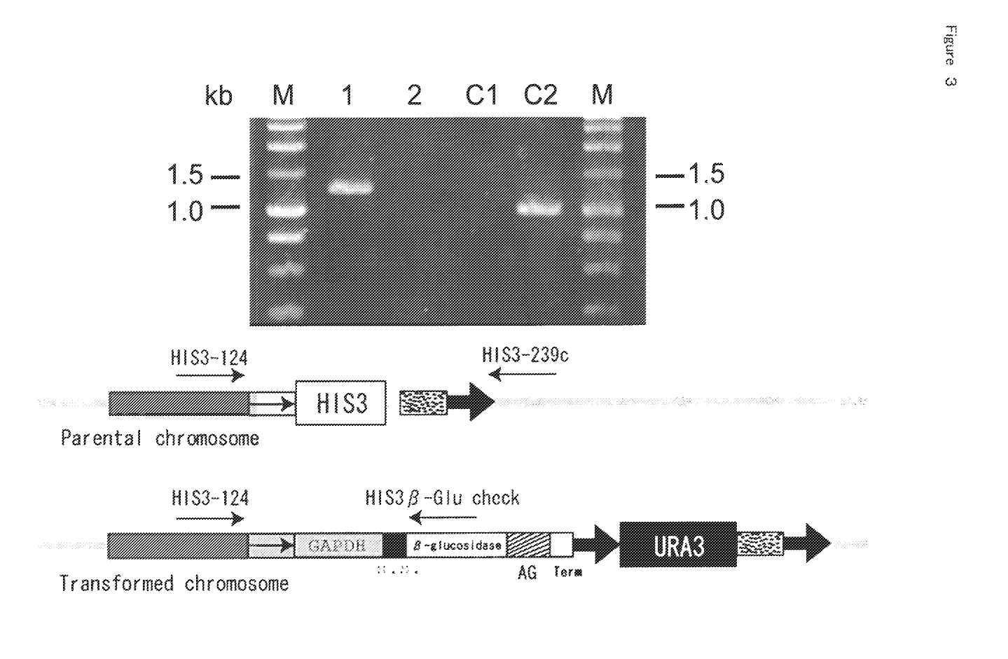

It was checked by colony PCR as to whether the disruption of the HIS3 gene and the integration of the expression cassette for β-glucosidase cell surface displaying gene had occurred or not in the resultant transformant. FIG. 3 shows an electrophoretogram showing the results of the colony PCR together with a schematic diagram showing the primers used in this colony PCR and the regions to be amplified therewith.

Regarding the presence of the HIS3 gene, a primer pair of HIS3-124 (SEQ ID No. 6; Forward) and HIS3-239c (SEQ ID No. 5; Reverse) was used to check whether or not amplification had occurred (the upper schematic diagram in FIG. 3). Regarding the presence of the expression cassette for a gene displaying β-glucosidase on the cell surface, a primer pair of HIS3-124 (SEQ ID No. 6; Forward) and HIS3 β-Glu check (SEQ ID No. 7; Reverse) was used to check whether or not amplification had occurred (the lower schematic diagram in FIG. 3).

In the electrophoretogram in FIG. 3, Lane 1 shows the results of the colony PCR of the transformant using the primer pair of HIS3-124 (SEQ ID No. 6; Forward) and HIS3 β-Glu check (SEQ ID No. 7; Reverse), Lane 2 shows the results of the colony PCR of the transformant using the primer pair of HIS3-124 (SEQ ID No. 6; Forward) and HIS3-239c (SEQ ID No. 5; Reverse), Lane C1 shows the results of the colony PCR of the NBRC1440 parental strain using the primer pair of HIS3-124 (SEQ ID No. 6; Forward) and HIS3 β-Glu check (SEQ ID No. 7), and Lane C2 shows the results of the colony PCR of the NBRC1440 parental strain using the primer pair of HIS3-124 (SEQ ID No. 6; Forward) and HIS3-239c (SEQ ID No. 5; Reverse). Lanes “M” at the both ends are indicators of the length of a formed fragment. While a 1.0 kb band was observed in the colony PCR of the NBRC1440 parental strain using the primer pair of HIS3-124 (SEQ ID No. 6; Forward) and HIS3-239c (SEQ ID No. 5; Reverse) (Lane C2), a 1.3 kb band was observed in the colony PCR of the transformant using the primer pair of HIS3-124 (SEQ ID No. 6; Forward) and HIS3 β-Glu check (SEQ ID No. 7; Reverse) (Lane 1). Accordingly, it was determined that the HIS3 gene was deleted (disrupted) and instead the expression cassette for β-glucosidase cell surface displaying gene was properly inserted.

The β-glucosidase activity of the transformant was measured to be 25.7 U/g (cell wet weight; average value of three independent experiments). This value was about 20% greater than the value 21.3 U/g of the laboratory strain MT8-1 (Non-Patent Document 10).

Subsequently, this transformant was grown in YPD medium at 30° C. for 24 hours and then spread 1.0×10⁷ cells/200 μL on a 5-FOA medium plate to. All the colonies grown on the 5-FOA medium plate were uracil auxotrophy (Ura⁻) phenotypes which was selected.

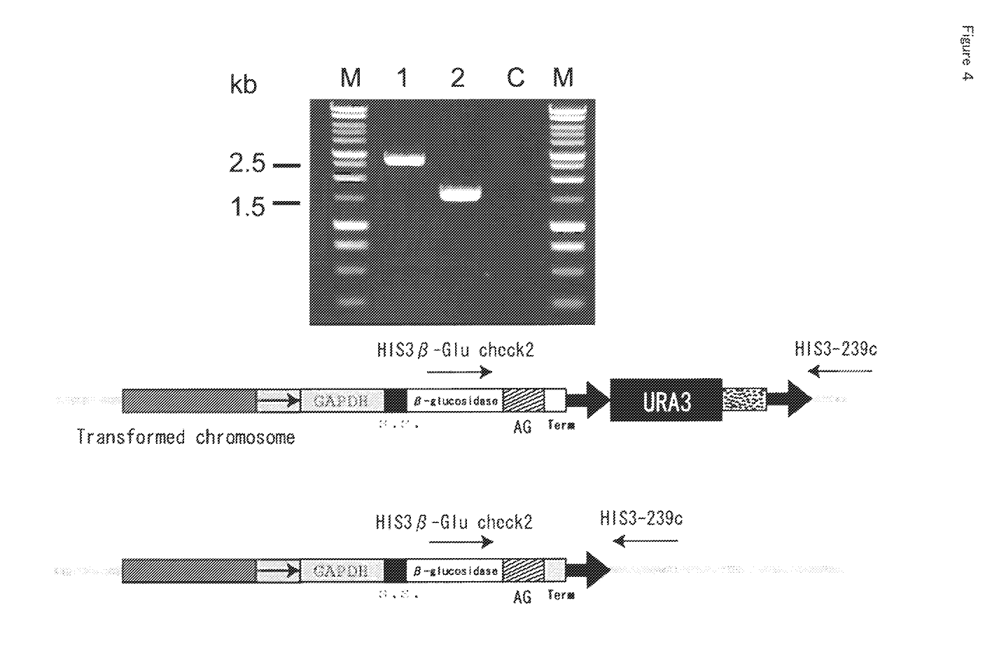

It was confirmed also in the colony PCR that the URA3 marker had been eliminated from the transformant acquired through selection using the 5-FOA medium. FIG. 4 shows an electrophoretogram showing the results of the colony PCR together with a schematic diagram showing primers used in this colony PCR and regions to be amplified therewith.

Colony PCR was performed using a primer pair of HIS3 β-Glu check2 (SEQ ID No. 8; Forward) and HIS3-239c (SEQ ID No. 5; Reverse) (the schematic diagrams in FIG. 4). In this colony PCR, the presence of the β-glucosidase gene on the chromosome and the elimination of the URA3 marker were checked.

In the electrophoretogram in FIG. 4, Lane 1 shows the result of the colony PCR of the transformant before the selection by the 5-FOA medium (uracil drop-out selected strain; no YPD growth was performed thereafter), Lane 2 shows the result of the colony PCR of the transformant after the selection by the 5-FOA medium (strain on which YPD growth was performed thereafter), and Lane C shows the result of the colony PCR of the NBRC1440 parental strain. Lanes “M” are indicators of the length of the resultant fragment. While a 2.7 kb band was observed in the colony PCR of the transformant before selection by the 5-FOA medium (Lane 1), a 1.6 kb band was observed in the colony PCR of the transformant after the selection by the 5-FOA medium (Lane 2) (no band was observed in Lane C). All the 5-FOA resistant colonies contained 40 bp of a repeat sequence and it was thus found that the URA3 marker had been actually eliminated.

From the results of the colony PCR, it could be determined that the URA3 marker had been eliminated but the β-glucosidase left in the transformant after the selection by the 5-FOA medium. In the transformant grown on the 5-FOA medium plate, due to the occurrence homologous recombination by the repeat sequences present one on each side of the URA3 marker (the schematic diagram shown immediately below the second downward-pointing arrow from the top in FIG. 2), the URA3 marker that had been introduced by the transformation was eliminated from the chromosome (the lowermost schematic diagram in FIG. 2), and the transformant exhibited the uracil auxotrophy (Ura⁻) phenotype.

Accordingly, with the 5-FOA selection, an industrial yeast strain in which the HIS3 gene and the URA3 gene had been deleted and the β-glucosidase gene had been integrated was acquired, which was designated “NBRC1440/HU-BGL13”.

Furthermore, in this example, the recombination frequency that occurs between 40 bp repeats was calculated as 3.6×10⁻⁶. This frequency was slightly higher than 2.9×10⁻⁶ reported in Non-Patent Document 3.

### Example 4

### Ethanol Fermentation from Cellobiose Using Transformed Industrial Yeast

The transformant (“NBRC1440/HU-BGL13”) eventually obtained in Example 3 was aerobically pre-cultured for 24 hours in SD medium added essential amino acids, and then cultured at 30° C. for 48 hours in YPD medium. The culture supernatant and cell pellets were separated by centrifugation at 6000×g for 10 minutes at 4° C. and the separated cell pellets were inoculated into YPCellobiose medium containing 50 g/L of cellobiose as the sole carbon source. The subsequent fermentation was performed anaerobically at 30° C. The initial cell concentration in the fermentation was adjusted to be 75 g/L (wet cells). Ethanol and cellobiose concentrations during fermentation were measured using HPLC. The HPLC analysis was performed using a refractive index (RI) detector (L-2490 RI detector, manufactured by Hitachi, Ltd.). The column used for separation was a Shim-pack SPR-Pb Column (manufactured by Shimadzu Corporation). HPLC was carried out at 80° C. by using water at a flow rate of 0.6 mL/min as the mobile phase.

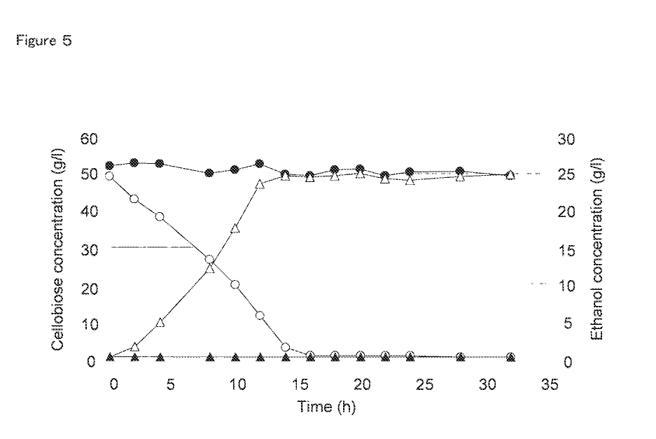

The results are shown in FIG. 5. FIG. 5 is a graph showing the time course of the amounts of cellobiose and ethanol during fermentation for NBRC1440/HU-BGL13 and NBRC1440. In FIG. 5, the right vertical axis represents ethanol concentration (g/L), the left vertical axis represents cellobiose concentration (g/L), and the horizontal axis represents elapsed time (hour). The black circles represent the cellobiose concentration in NBRC1440, the white circles represent the cellobiose concentration in NBRC1440/HU-BGL13, the black triangles represent the ethanol concentration in NBRC1440, and the white triangles indicate the ethanol concentration in NBRC1440/HU-BGL13. As shown in FIG. 5, NBRC1440/HU-BGL13 simultaneously hydrolysed cellobiose to glucose and fermented the produced glucose to ethanol.

The 50 g/L of cellobiose was completely depleted in 16 hours in 75 g/L of cell concentration, and the yield of ethanol was 0.49 g/g of cellobiose consumed. This corresponds to 96% of the theoretical yield (calculated that 0.51 g of ethanol was produced per g of saccharide consumed), which was calculated by the equation of the Embden-Meyerhof pathway. During fermentation, glucose did not accumulate in the medium.

### Example 5

### Construction of Chromosomal Integrating Plasmid for Cellobiohydrolase Surface Display

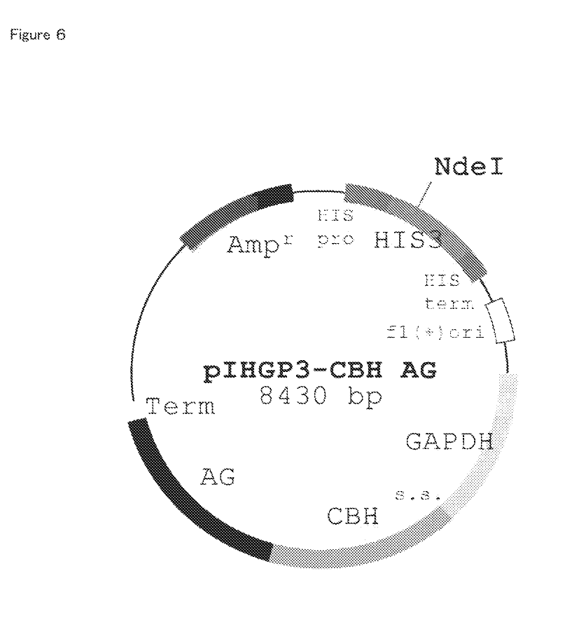

A plasmid pIHGP3-CBH2 was constructed, which contains a histidine gene (HIS3) marker and which is for integrating cellobiohydrolase (CBH2) gene such that CBH2 can be displayed on cell surface. FIG. 6 shows a schematic diagram of this plasmid. In FIG. 6, the “HIS3” represents the histidine gene, “HIS3 pro” represents HIS3 promoter, “HIS3 term” represents HIS3 terminator, “GAPDH pro” represents glyceraldehyde-3-phosphate dehydrogenase promoter, “s.s.” represents the secretory signal sequence of Rhizopus oryzae-derived glucoamylase gene, “CBH2” represents Trichoderma reesei-derived cellobiohydrolase 2 gene, and “GAPDH term” represents glyceraldehyde-3-phosphate dehydrogenase terminator.

A 2816 kbp DNA fragment coding for the secretory signal sequence of Rhizopus oryzae-derived glucoamylase gene, cbh2 gene, and the 3′ half of α-agglutinin gene was prepared through PCR with a primer pair of R.ory s.s. XmaI (SEQ ID No. 15; Forward) and CBH XbaI Rev (SEQ ID No. 16; Reverse) using pFCBH2w3 (Non-Patent Document 11) as a template.

A primer pair of XYL2c-XhoI (F) (SEQ ID No. 17; Forward) and XYL2c-NotI (R) (SEQ ID No. 18; Reverse) was used to perform PCR amplification of the genetic sequence coding for GAPDH promoter, a multi-cloning site (SalI, XbaI, BamHI, SmaI, XmaI), and GAPDH terminator using pUGP3 (Non-Patent Document 12) as a template. This fragment was introduced into the XhoI/NotI site of pRS403 (Stratagene) to obtain a plasmid pIHGP3.

The 2816 kbp DNA fragment was digested with XmaI and XbaI and inserted between the XmaI site and the XbaI site of plasmid pIHGP3 containing GAPDH promoter and GAPDH terminator to obtain a plasmid which contains HIS3 gene and its promoter and terminator, GAPDH promoter, a gene coding for the secretory signal sequence of glucoamylase gene, cellobiohydrolase (CBH2) gene, the 3′ half of α-agglutinin gene, and GAPDH terminator. The obtained plasmid was designated pIHGP3-CBH2 AG (FIG. 6).

### Example 6

### Construction of Chromosomal Integrating Plasmid for Endoglucanase Surface Display

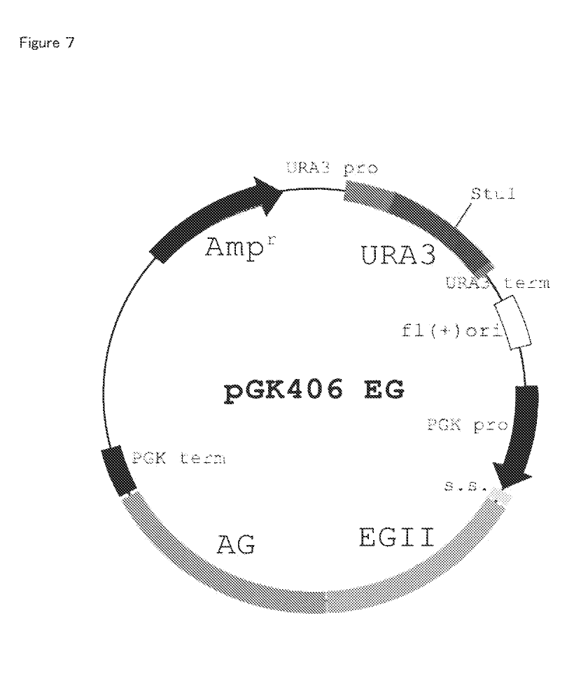

A plasmid pGK406 EG was constructed, which contains an uracil gene (URA3) marker and which is for integrating endoglucanase (EGII) gene such that EGII can be displayed on cell surface. FIG. 7 shows a schematic diagram of this plasmid. In FIG. 7, the “URA3” represents the uracil gene, “URA3 pro” represents URA3 promoter, “URA3 term” represents a URA3 terminator, “PGK pro” represents phosphoglycerate kinase promoter, “s.s.” represents the secretory signal sequence of Rhizopus oryzae-derived glucoamylase gene, “AG” represents the 3′ half of α-agglutinin gene, and “PGK term” represents phosphoglycerate kinase terminator.

A 2719 kbp DNA fragment for encoding the secretory signal sequence of Rhizopus oryzae-derived glucoamylase gene, EG2 gene, and the 3′ half of α-agglutinin gene was prepared through PCR with a primer pair of EG NheI Fw (SEQ ID No. 19; Forward) and EG XmaI Rev (SEQ ID No. 20; Reverse) using pEG23u31H6 (Non-Patent Document 10) as a template.

Two DNA fragments of PGK promoter and PGK terminator, respectively were amplified through PCR with a primer pair for PGK promoter (SEQ ID No. 21; Forward and SEQ ID No. 22; Reverse) and a primer pair for PGK terminator (SEQ ID No. 23; Forward and SEQ ID No. 24; Reverse), which were designed respectively for PGK promoter and PGK terminator, using the genome DNA of Saccharomyces cerevisiae BY4741 as a template. A multi-cloning site was prepared by annealing a designed primer pair for a multi-cloning site (SEQ ID No. 25; Forward and SEQ ID No. 26; Reverse). The PGK promoter was digested with XhoI and NheI, the multi-cloning site was digested with NheI and BglII, and the PGK terminator was digested with BglII and Nod, and then, they were cloned into the XhoI-NotI site of pTA2 vector (manufactured by Toyobo Co. Ltd., Osaka, Japan). The resultant vector was digested with XhoI and Nod, and the resultant fragment was cloned in pRS406 (Stratagene), to obtain a vector which was designated pGK406.

the 2719 kbp DNA fragment was digested with NheI and XmaI, and inserted between the NheI site and the XmaI site of plasmid pGK406 which contains the URA3 gene and its promoter and terminator, PGK promoter, and PGK terminator to obtain a plasmid containing a URA3 gene, and its promoter and terminator, PGK promoter, a gene coding for the secretory signal sequence of glucoamylase gene, endoglucanase (EGII) gene, the 3′ half of α-agglutinin gene, and a PGK terminator. The obtained plasmid was designated pGK406 EG (FIG. 7).

### Example 7

### Production of Transformed Industrial Yeast Displaying Three Types of Cellulase on Cell Surface

The plasmid PGK406 EG prepared according to Example 6 was digested with restriction enzyme StuI into a linear form, and was transformed into NBRC1440/HU-BGL13 (a transformant eventually obtained in Example 3, an industrial yeast strain in which the HIS3 gene and the URA3 gene had been deleted and the β-glucosidase gene had been integrated), and a strain having no uracil auxotrophy was selected on an uracil drop-out plate (an uracil-free medium). Following the transformation with pGK406 EG, the disrupted URA3 marker gene of the NBRC1440/HU-BGL13 was restored and thus the introduction of the gene was confirmed. This strain was designated “NBRC1440/HU-BGL13/pGK406 EG”.

Furthermore, the plasmid pIHGP3-CBH2 AG prepared according to Example 5 was digested with restriction enzyme NdeI into a linear form, and was transformed into NBRC1440/HU-BGL13/pGK406 EG, and a strain having neither histidine nor uracil auxotrophy was selected on an histidine-uracil drop-out plate (a medium not containing histidine and uracil). In a similar manner, following the transformation using pIHGP3-CBH2 AG, the disrupted HIS3 marker gene of NBRC1440/HU-BGL13/pGK406 EG was restored and thus the introduction of the gene was confirmed. This strain was designated “NBRC1440/HU-BGL13/pIHGP3-CBH AG/pGK406 EG”.

### Example 8

### Ethanol Fermentation from Cellulose Using Transformed Industrial Yeast

The transformed industrial yeasts NBRC1440/HU-BGL13/pIHGP3-CBH AG/pGK406 EG, NBRC1440/HU-BGL13/pGK406 EG, and NBRC1440/HU-BGL13 were used to compare ethanol fermentation from cellulose.

The yeasts were aerobically pre-cultured for 24 hours in SD medium added essential amino acids, and then cultured at 30° C. for 48 hours in a YPD medium. The culture supernatant and cell pellets were separated by centrifugation at 6000×g for 10 minutes at 4° C. and thus cell pellets were obtained.

These cell pellets were inoculated into a fermentation medium containing 10 g/L of phosphoric acid-swollen cellulose, 20 g/L of yeast extract, 10 g/L of polypeptone, 50 mM of citrate buffer (pH 5.0), and 0.05% (w/v) potassium metabisulfite. The subsequent fermentation was performed anaerobically at 30° C. The initial cell concentration in the fermentation adjusted to be 75 g/L (wet cells). Ethanol concentration during fermentation was measured using HPLC. HPLC was performed using a refractive index (RI) detector (L-2490 RI detector, manufactured by Hitachi, Ltd.). The column used for separation was a Shim-pack SPR-Pb Column (manufactured by Shimadzu Corporation). The HPLC analysis was carried out at 80° C. using water at a flow rate of 0.6 mL/min as the mobile phase.

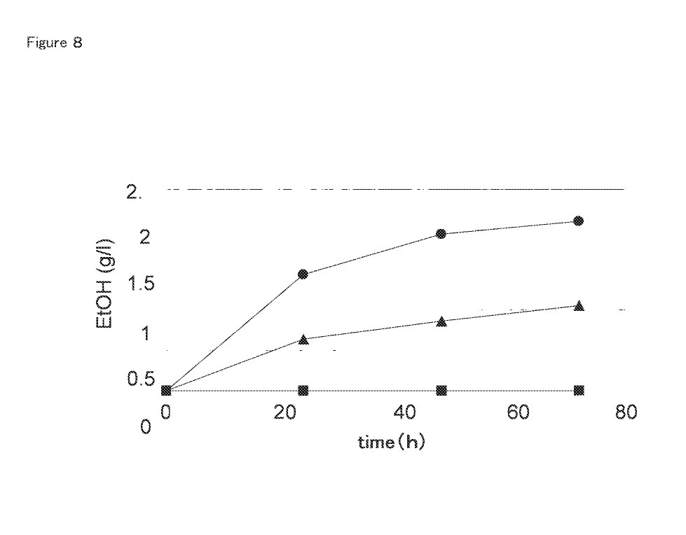

The results are shown in FIG. 8. FIG. 8 is a graph showing the time course of the amount of ethanol produced during fermentation from cellulose for NBRC1440/HU-BGL13/pIHGP3-CBH AG/pGK406 EG, NBRC1440/HU-BGL13/pGK406 EG, and NBRC1440/HU-BGL13. In FIG. 8, the left vertical axis represents ethanol concentration (g/L) and the horizontal axis represents elapsed time (hour). The black circles represent the ethanol concentration in NBRC1440/HU-BGL13/pIHGP3-CBH AG/pGK406 EG, the black triangles represent the ethanol concentration in NBRC1440/HU-BGL13/pGK406 EG, and the black squares indicate the ethanol concentration in NBRC1440/HU-BGL13. In particular, the transformed industrial yeast which displays three types of cellulase on cell surface NBRC1440/HU-BGL13/pIHGP3-CBH AG/pGK406 EG exhibited the greatest ethanol production (FIG. 8).

Accordingly, this example showed that an industrial yeast that has an ability to degrade cellulose and that is capable of producing ethanol from cellulose was obtained.

### INDUSTRIAL APPLICABILITY

According to the present invention, a foreign gene can be introduced into an industrial yeast that is desirable in alcohol production but on which it is difficult to perform gene manipulation due to its lack of auxotrophy. The method of the present invention simultaneously provides an auxotrophic marker to and introduces a foreign gene into an yeast, and it is therefore possible to introduce a further foreign gene using the provided marker. For example, the method of the present invention can produce a transformed industrial yeast that displays three types of cellulase on the surface, thus allowing for ethanol production from plant biomass even under the culture in industrial scale which may be performed under severe culturing conditions.

1. A method for providing a target auxotrophy to and introducing a gene to be expressed into a yeast cell, comprising:
(a) transforming, with a fragment comprising an expression cassette for the gene to be expressed, a cassette for a yeast selectable marker that controls an auxotrophy different from the target auxotrophy and that allows counter selection, and two regions for homologous recombination, a yeast cell to which a marker of the different auxotrophy is provided,
wherein, in the fragment,
an upstream-side one of the homologous recombination regions is homologous to a region upstream (on the side of 5′ end) of a gene controlling the target auxotrophy in the yeast cell and is arranged upstream (on the side of 5′ end) of the expression cassette, and a downstream-side one of the homologous recombination regions is homologous to a region downstream (on the side of 3′ end) of the gene controlling the target auxotrophy and is arranged downstream (on the side of 3′ end) of the expression cassette,
the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the gene controlling the target auxotrophy, and
the yeast selectable marker cassette is arranged between the expression cassette and the downstream-side one of the homologous recombination region,
thereby causing a first homologous recombination;
(b) selecting for a transformed yeast not having the different auxotrophy, the transformed yeast from which the gene controlling the target auxotrophy is deleted and into which the gene to be expressed and the yeast selectable marker cassette is introduced;
(c) causing a second homologous recombination, in the transformed yeast selected in step (b), between the repeat region and the region further downstream (on the side of 3′ end) of the region downstream (on the side of 3′ end) of the gene controlling the target auxotrophy; and
(d) selecting for a transformed yeast that has acquired the different auxotrophy, to obtain a transformed yeast from which the gene controlling the target auxotrophy and the gene controlling the different auxotrophy are deleted, and which has an auxotrophy therefor, and into which the gene to be expressed is introduced.

2. A method according to claim 1, further comprising providing the different auxotrophy to the yeast cell.

3. A method according to claim 1, wherein the different auxotrophy gene marker is uracil auxotrophy.

4. A method for repetitively introducing a gene into a yeast cell, comprising:
further transforming a transformed yeast cell produced according to the method of claim 1, from which an auxotrophy-controlling gene is deleted and into which a gene to be expressed is introduced, with a vector comprising the auxotrophy-controlling gene deleted from the transformed yeast cell and an additional gene to be expressed.

5. A vector for providing a target auxotrophy to and introducing a gene to be expressed into a yeast cell, comprising:
a fragment comprising an expression cassette for the gene, a cassette for a yeast selectable marker that is a gene controlling an auxotrophy different from the target auxotrophy, and two regions for homologous recombination,
wherein, in the fragment,
an upstream-side one of the homologous recombination regions is homologous to a region upstream (on the side of 5′ end) of a gene controlling the target auxotrophy in the yeast cell and is arranged upstream (on the side of 5′ end) of the expression cassette, and a downstream-side one of the homologous recombination regions is homologous to a region downstream (on the side of 3′ end) of the gene controlling the target auxotrophy and is arranged downstream (on the side of 3′ end) of the expression cassette,
the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the gene controlling the target auxotrophy, and
the yeast selectable marker cassette is arranged between the expression cassette and the downstream-side one of the homologous recombination regions.

6. A vector according to claim 5, wherein the different auxotrophy is uracil auxotrophy.

7. A method for introducing a gene to be expressed into a yeast cell, the method comprising:
(i) transforming the yeast cell with a fragment comprising an expression cassette for the gene to be expressed, a cassette for a yeast selectable marker, and two regions for homologous recombination,
wherein, in the fragment,
an upstream-side one of the homologous recombination regions is homologous to a region upstream (on the side of 5′ end) of a target locus in the yeast cell and is arranged upstream (on the side of 5′ end) of the expression cassette, and a downstream-side one of the homologous recombination regions is homologous to a region downstream (on the side of 3′ end) of the target locus and is arranged downstream (on the side of 3′ end) of the expression cassette,
the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the target locus to which the downstream-side one of the homologous recombination regions is homologous, and
the yeast selectable marker cassette is arranged between the expression cassette and the downstream-side one of the homologous recombination regions,
thereby causing a first homologous recombination, and wherein while the target locus is deleted, the gene to be expressed and the yeast selectable marker cassette is introduced into the yeast cell; and
(ii) causing a second homologous recombination between the region further downstream (on the side of 3′ end) of the region downstream (on the side of 3′ end) of the target locus and the repeat region to delete the yeast selectable marker from the transformed yeast cell.

8. A vector for introducing a gene to be expressed into a yeast cell, the vector comprising:
a fragment comprising an expression cassette for the gene to be expressed, a cassette for a yeast selectable marker, and two regions for homologous recombination,
wherein in the fragment,
an upstream-side one of the homologous recombination regions is homologous to a region upstream (on the side of 5′ end) of a target locus in the yeast cell and is arranged upstream (on the side of 5′ end) of the expression cassette, and a downstream-side one of the homologous recombination regions is homologous to a region downstream (on the side of 3′ end) of the target locus and is arranged downstream (on the side of 3′ end) of the expression cassette,
the yeast selectable marker cassette comprises the yeast selectable marker and, upstream (on the side of 5′ end) of the yeast selectable marker, a repeat region that is homologous to a region further downstream (on the side of 3′ end) of the region downstream (on the side of 3′ end) of the target locus to which the downstream-side one of the homologous recombination regions is homologous, and
the yeast selectable marker cassette is arranged between the expression cassette and the downstream-side one of the homologous recombination regions.

9. A cellulolytic industrial yeast recombined to express an enzyme that can cleave a β-1,4-glycosidic bond.

10. A cellulolytic industrial yeast according to claim 9, wherein the enzyme that can cleave a β-1,4-glycosidic bond is a combination of β-glucosidase, endoglucanase, and cellobiohydrolase.

11. A method for producing ethanol, comprising:
reacting the cellulolytic industrial yeast of claim 9 with a cellulose-based material to yield ethanol.

12. A method according to claim 2, wherein the different auxotrophy gene marker is uracil auxotrophy.

13. A method for repetitively introducing a gene into a yeast cell, comprising:
further transforming a transformed yeast cell produced according to the method of claim 2, from which an auxotrophy-controlling gene is deleted and into which a gene to be expressed is introduced, with a vector comprising the auxotrophy-controlling gene deleted from the transformed yeast cell and an additional gene to be expressed.

14. A method for repetitively introducing a gene into a yeast cell, comprising:
further transforming a transformed yeast cell produced according to the method of claim 3, from which an auxotrophy-controlling gene is deleted and into which a gene to be expressed is introduced, with a vector comprising the auxotrophy-controlling gene deleted from the transformed yeast cell and an additional gene to be expressed.

15. A method for producing ethanol, comprising:
reacting the cellulolytic industrial yeast of claim 10 with a cellulose-based material to yield ethanol.


SEQUENCE LISTING (26 sequences)

SEQ ID NO: 1 - DNA, 30 bp, Artificial
  Primer for HIS3-966 XbaI
gctctagacccgagaggaaacttcttagcg

SEQ ID NO: 2 - DNA, 24 bp, Artificial
  Primer for HIS3-1c
ctttgccttcgtttatcttgcctg

SEQ ID NO: 3 - DNA, 89 bp, Artificial
  Primer for HIS3URA3-2 AscI
caggcaagataaacgaaggcaaagggcgcgccatcattctatacgtgtcattctgaacgaggcgcgctttccgattcggtaatctccga

SEQ ID NO: 4 - DNA, 68 bp, Artificial
  Primer for HIS3-40Uc XbaI
gctctagagcattaccttgtcatcttcagtatcatactgttcgtatacgggtaataactgatataatt

SEQ ID NO: 5 - DNA, 22 bp, Artificial
  Primer for HIS3-239c
ctttttcccgttcctccatctc

SEQ ID NO: 6 - DNA, 21 bp, Artificial
  Primer for HIS3-124
cttagcgattggcattatcac

SEQ ID NO: 7 - DNA, 24 bp, Artificial
  Primer for HIS3 fA-Glu check
gagaacgccagttcatcagagatc

SEQ ID NO: 8 - DNA, 24 bp, Artificial
  Primer for HIS3 fA-Glu check2
gaagaaggtccatgttggtagctc

SEQ ID NO: 9 - DNA, 34 bp, Artificial
  Primer for GAPDH AscI
ttggcgcgccaccagttctcacacggaacaccac

SEQ ID NO: 10 - DNA, 43 bp, Artificial
  Primer for GAPDH AscI Rev
ttggcgcgccctattttaatgacattttcgattcattgattga

SEQ ID NO: 11 - DNA, 40 bp, Artificial
  bgl1 primer 1
gatctccatggctgatgaactggcgttctctcctcctttc

SEQ ID NO: 12 - DNA, 40 bp, Artificial
  bgl1 primer 2
tggcgctcgagccttgcaccttcgggagcgccgcgtgaag

SEQ ID NO: 13 - DNA, 22 bp, Artificial
  Primer for URA3 delete
ggagaatccatacaagaaatcg

SEQ ID NO: 14 - DNA, 26 bp, Artificial
  Primer for URA3 delta r
gtaaaatactgttacttggttctggc

SEQ ID NO: 15 - DNA, 38 bp, Artificial
  Primer for R.ory s.s. XmaI
tccccccgggatgcaactgttcaatttgccattgaaag

SEQ ID NO: 16 - DNA, 43 bp, Artificial
  Primer for CBH XbaI Rev
gggtctagatttgattatgttctttctatttgaatgagatatg

SEQ ID NO: 17 - DNA, 40 bp, Artificial
  Primer for XYL2c-XhoI(F)
ccgctcgagaccagttctcacacggaacaccactaatgga

SEQ ID NO: 18 - DNA, 45 bp, Artificial
  Primer for XYL2c-NotI(R)
atagtttagcggccgctcaatcaatgaatcgaaaatgtcattaaa

SEQ ID NO: 19 - DNA, 37 bp, Artificial
  Primer for EG NheI Fw
ctagctagcatgcaactgttcaatttgccattgaaag

SEQ ID NO: 20 - DNA, 44 bp, Artificial
  Primer for EG XmaI Rev
tccccccgggtttgattatgttctttctatttgaatgagatatg

SEQ ID NO: 21 - DNA, 30 bp, Artificial
  Primer 1 for PGK promoter of pGK406
ttttctcgagaaagatgccgatttgggcgc

SEQ ID NO: 22 - DNA, 30 bp, Artificial
  Primer 2 for PGK promoter of pGK406
gcccgctagcgttttatatttgttgtaaaa

SEQ ID NO: 23 - DNA, 30 bp, Artificial
  Primer 1 for PGK terminator of pGK406
gcccagatctgaaataaattgaattgaatt

SEQ ID NO: 24 - DNA, 30 bp, Artificial
  Primer 2 for PGK terminator of pGK406
ttttgcggccgcagctttaacgaacgcaga

SEQ ID NO: 25 - DNA, 52 bp, Artificial
  Primer 1 for multi-cloning site of pGK406
ttttgctagcgtcgacactagtggatcccccgggtctagagaattcagatct

SEQ ID NO: 26 - DNA, 52 bp, Artificial
  Primer 2 for multi-cloning site of pGK406
ttttagatctgaattctctagacccgggggatccactagtgtcgacgctagc